(19) United States
(12) Patent Application Publication
(10) Pub. No.: US 2022/0032020 A1
(43) Pub. Date: Feb. 3, 2022

(54) THERAPEUTIC SUBSTANCE STORAGE AND DELIVERY

(71) Applicant: Cochlear Limited, Macquarie University, NSW (AU)

(72) Inventor: Daniel SMYTH, Macquarie University (AU)

(21) Appl. No.: 17/280,291

(22) PCT Filed: Dec. 4, 2019
(86) PCT No.: PCT/IB2019/060429

§ 371 Date: Mar. 26, 2021

Related U.S. Application Data
(60) Provisional application No. 62/775,645, filed on Dec. 5, 2018.

(51) Int. Cl. A61M 31/00

(52) U.S. Cl. CPC A61M 31/00; A61M 2210/0668

(57) ABSTRACT

An apparatus, including a refillable therapeutic substance delivery device including a reservoir, the reservoir being configured to be located in an adult middle ear cavity of a human recipient, wherein in some instances, the device is configured such that the reservoir is accessible through a tympanic membrane of the recipient.

### CROSS-REFERENCE TO RELATED APPLICATIONS

This application claims priority to U.S. Provisional Application No. 62/775,645, entitled THERAPEUTIC SUBSTANCE STORAGE AND DELIVERY, filed on Dec. 5, 2018, naming Daniel SMYTH of Mechelen, Belgium as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve. Cases of conductive hearing loss typically are treated by means of bone conduction hearing aids. In contrast to conventional hearing aids, these devices use a mechanical actuator that is coupled to the skull bone to apply the amplified sound.

In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

It is also noted that the electrode array of the cochlear implant generally shows utilitarian results if it is inserted in a cochlea.

### SUMMARY

In accordance with an exemplary embodiment, there is an apparatus, comprising a refillable therapeutic substance delivery device including a reservoir, the reservoir being configured to be located in a middle ear cavity of a human recipient.

In accordance with another exemplary embodiment, there an apparatus, comprising a refillable therapeutic substance delivery device securable to a round window niche of a recipient.

In accordance with another exemplary embodiment, there an apparatus, comprising a means for refillably storing a therapeutic substance and a means for delivering a therapeutic substance to a cochlea.

In accordance with another exemplary embodiment, there is a method, comprising obtaining access to a middle ear cavity of a person, inserting a therapeutic substance delivery device into the middle ear cavity through an ear canal of the person and securing the therapeutic substance delivery device in the middle ear cavity such that the therapeutic substance delivery device delivers therapeutic substance to the cochlea from a storage location in the middle ear cavity.

### BRIEF DESCRIPTION OF THE DRAWINGS

Embodiments are described below with reference to the attached drawings, in which:

FIG. 1 is a perspective view of an exemplary hearing prosthesis;

FIGS. 2A-2H are views of exemplary electrode arrays to which the teachings detailed herein can be applicable;

FIGS. 3A and 3B are side and perspective views of an electrode assembly extended out of an embodiment of an insertion sheath of the insertion tool illustrated in FIG. 2;

FIGS. 4A-4E are simplified side views depicting an exemplary insertion process of the electrode assembly into the cochlea;

FIGS. 5-7 present an exemplary therapeutic substance delivery system;

FIG. 8 presents an exemplary embodiment of an exemplary therapeutic substance delivery system implanted in a recipient;

FIGS. 9 and 10 and 13 present exemplary embodiments of the system of FIG. 8;

FIGS. 11 and 12 and 14 present exemplary scenarios of utilization of the system;

FIG. 15 presents an exemplary alternate embodiment of mounting the therapeutic substance delivery device in a middle ear cavity;

FIG. 16 presents an alternate embodiment of the reservoir;

FIGS. 17-19 present exemplary flowcharts for exemplary methods;

FIGS. 20 and 21 present exemplary embodiments associated with placements of the delivered device in a middle ear;

FIG. 21 presents another exemplary embodiment of the delivery device;

FIGS. 22-25 present alternate exemplary embodiments for vibrationally isolating the delivery device from the tympanic membrane;

FIGS. 26 and 27 present exemplary embodiments of transferring therapeutic substance from inside the reservoir to outside the reservoir; and

FIG. 28 presents an exemplary pump according to an exemplary embodiment.

### DETAILED DESCRIPTION

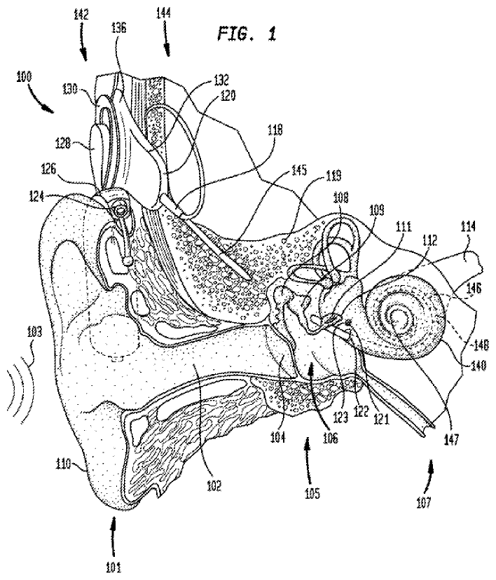

FIG. 1 is a perspective view of an exemplary cochlear implant 100 implanted in a recipient having an outer ear 101, a middle ear 105, and an inner ear 107. In a fully functional ear, outer ear 101 comprises an auricle 110 and an ear canal 102. It is briefly noted that while some embodiments focused upon a cochlear implant that utilizes an electrode array, other embodiments can be utilized in combination with other types of implants, such as, for example, a middle ear implant or a direct acoustic cochlear stimulation device. Embodiments include the utilization of the teachings detailed herein with a mechanical actuator device that is located inside the cochlea. Moreover, at least some exemplary embodiments can be utilized with transcutaneous bone conduction devices and/or conventional acoustic hearing aids that are entirely outside the skin of the recipient. Any disclosure of one herein corresponds to a disclosure of any one or more of the others unless otherwise noted.

Acoustic pressure or sound waves 103 are collected by auricle 110 and channeled into and through ear canal 102. Disposed across the distal end of ear canal 102 is a tympanic membrane 104 that vibrates in response to sound waves 103. This vibration is coupled to oval window or fenestra ovalis 112 through the three bones of the middle ear 105, collectively referred to as the ossicles 106, and comprising the malleus 108, the incus 109, and the stapes 111. Ossicles 106 filter and amplify the vibrations delivered by tympanic membrane 104, causing oval window 112 to articulate, or vibrate. This vibration sets up waves of fluid motion of the perilymph within cochlea 140. Such fluid motion, in turn, activates hair cells (not shown) inside the cochlea which in turn causes nerve impulses to be generated which are transferred through spiral ganglion cells (not shown) and auditory nerve 114 to the brain (also not shown) where they are perceived as sound.

The exemplary cochlear implant illustrated in FIG. 1 is a partially-implanted stimulating medical device. Specifically, cochlear implant 100 comprises external components 142 attached to the body of the recipient, and internal or implantable components 144 implanted in the recipient. External components 142 typically comprise one or more sound input elements for detecting sound, such as microphone 124, a sound processor (not shown), and a power source (not shown). Collectively, these components are housed in a behind-the-ear (BTE) device 126 in the example depicted in FIG. 1. External components 142 also include a transmitter unit 128 comprising an external coil 130 of a transcutaneous energy transfer (TET) system. Sound processor 126 processes the output of microphone 124 and generates encoded stimulation data signals which are provided to external coil 130.

Internal components 144 comprise an internal receiver unit 132 including a coil 136 of the TET system, a stimulator unit 120, and an elongate stimulating lead assembly 118. Internal receiver unit 132 and stimulator unit 120 are hermetically sealed within a biocompatible housing commonly referred to as a stimulator/receiver unit. Internal coil 136 of receiver unit 132 receives power and stimulation data from external coil 130. Stimulating lead assembly 118 has a proximal end connected to stimulator unit 120, and extends through mastoid bone 119. Lead assembly 118 has a distal region, referred to as electrode assembly 145, a portion of which is implanted in cochlea 140.

Electrode assembly 145 can be inserted into cochlea 140 via a cochleostomy 122, or through round window 121, oval window 112, promontory 123, or an opening in an apical turn 147 of cochlea 140. Integrated in electrode assembly 145 is an array 146 of longitudinally-aligned and distally extending electrode contacts 148 for stimulating the cochlea by delivering electrical, optical, or some other form of energy. Stimulator unit 120 generates stimulation signals each of which is delivered by a specific electrode contact 148 to cochlea 140, thereby stimulating auditory nerve 114.

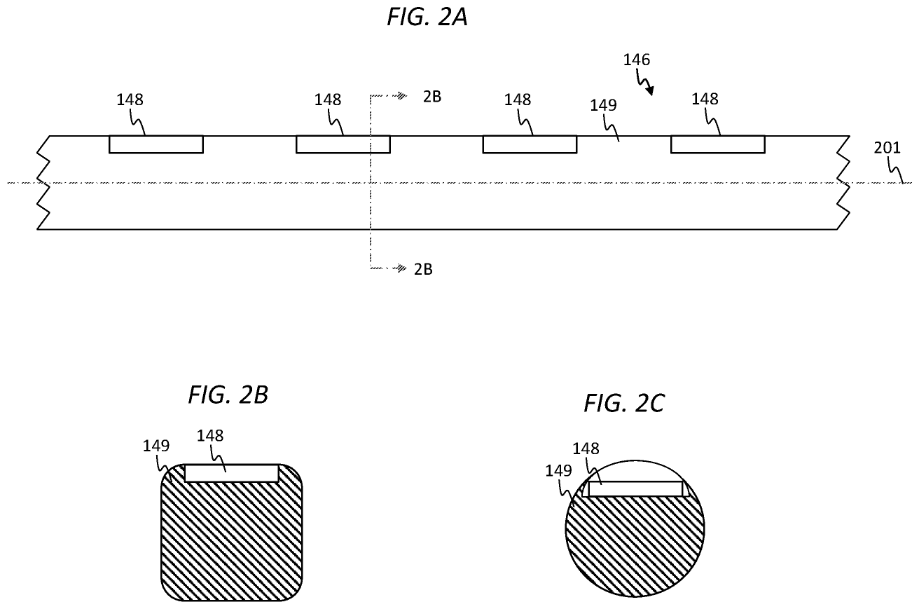

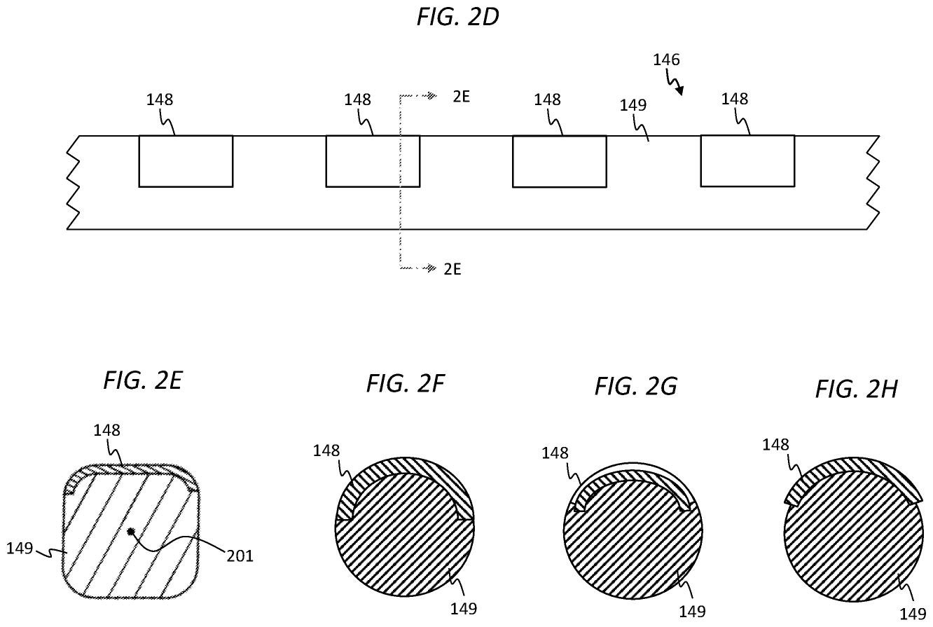

FIG. 2A depicts a conceptual side view of a portion of electrode array 146, depicting four electrode contacts 148 evenly spaced along a longitudinal axis of the electrode array 146. It is noted that in some alternate embodiments, the electrode is not evenly spaced. FIG. 2B depicts a conceptual cross-sectional view through one of the electrode contacts 148, which also depicts the carrier 149 of the electrode contact 148. In an exemplary embodiment, the carrier 149 is made of silicone. Not depicted in the figures are electrical leads and stiffener components that are sometimes embedded in the carrier 149. The embodiment of FIG. 2B represents an electrode array 146 that has a generally rectangular cross-section. FIG. 2C depicts an alternate embodiment where the electrode array 146 has a generally circular cross-section. It is also noted that in some exemplary embodiments, the cross-section is oval shaped. Thus, the embodiment of FIGS. 2A-2C is a species of the genus of an electrode array having a generally continuously curving cross-section. Any electrode array of any cross-section or any configuration can be utilized with the teachings detailed herein.

The electrode contacts 148 depicted in FIGS. 2A-2C are so-called flat contacts. In this regard, the surface of the electrode contact that faces the wall of the cochlea/the faces away from the longitudinal axis of the electrode array 146 is flat. Conversely, as seen in FIGS. 2D-2H, in some alternate embodiments, the electrode contacts 148 are so-called half band electrodes. In some exemplary embodiments, a band of contact material is “smashed” or otherwise compressed into a “half band,” as seen in the figures. It is noted that by “half band,” this does not mean that the electrode contact must necessarily span half of the outside diameter of the electrode array, as is the case in FIGS. 2G and 2H. The term is directed towards the configuration of the electrode itself as that term has meaning in the art. Any electrode contact that can have utilitarian value according to the teachings detailed herein can be utilized in at least some exemplary embodiments.

As can be seen from FIGS. 2A-2H, the positioning of the electrode contacts relative to the carrier 149 can vary with respect to alignment of the outer surface of the carrier with the outer surface of the contact. For example, FIGS. 2A, 2E, and 2F depict the outer surface of the contacts 148 as being flush with the outer surface of the carrier 149. Conversely, FIGS. 2C and 2G depict the contact 148 as being recessed with respect to the outer surface of the carrier 149, while FIG. 2H depicts the contact 148 as being proud relative to the outer surface of the contact 149. It is noted that these various features are not limited to the specific contact geometry and/or the specific carrier geometry depicted in the figures, and that one or more features of one exemplary embodiment can be combined with one or more features of another exemplary embodiment. For example, while FIG. 2H depicts a half band contact as being proud of the carrier 149 having a generally circular cross-section, a flat electrode such as that depicted in FIG. 2A can be proud of the carrier as well.

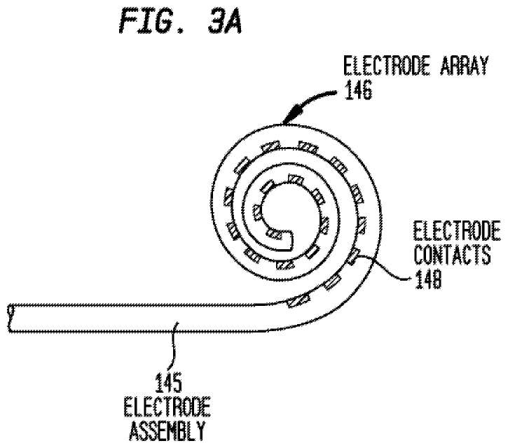

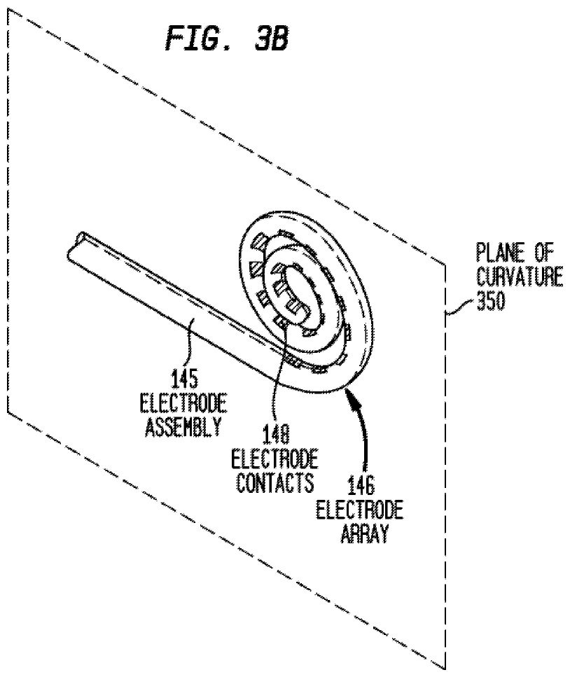

FIGS. 3A and 3B are side and perspective views, respectively, of representative electrode assembly 145. As noted, electrode assembly 145 comprises an electrode array 146 of electrode contacts 148. Electrode assembly 145 is configured to place electrode contacts 148 in close proximity to the ganglion cells in the modiolus. Such an electrode assembly, commonly referred to as a perimodiolar electrode assembly, is manufactured in a curved configuration as depicted in FIGS. 3A and 3B. When free of the restraint of a stylet or insertion guide tube, electrode assembly 145 takes on a curved configuration due to it being manufactured with a bias to curve, so that it is able to conform to the curved interior of cochlea 140. As shown in FIG. 3B, when not in cochlea 140, electrode assembly 145 generally resides in a plane 350 as it returns to its curved configuration. That said, it is noted that the teachings detailed herein and/or variations thereof can be applicable to a so-called straight electrode array, which electrode array does not curl after being free of a stylet or insertion guide tube etc., but instead remains straight. It is noted that when in the cochlea, the electrode assembly 145 takes on a conical shape with respect to plane 350 in that it can be described as winding upward away from the plane 350 about an axis normal thereto, owing to the shape of the cochlea (more on this below).

The perimodiolar electrode assembly 145 of FIGS. 3A and 3B is pre-curved in a direction that results in electrode contacts 148 being located on the interior of the curved assembly, as this causes the electrode contacts to face the modiolus when the electrode assembly is implanted in or adjacent to cochlea 140.

It is also noted that while the embodiments of FIGS. 2A-3B have been presented in terms of a so-called non-tapered electrode array (where the cross-sections of the array on a plane normal to the longitudinal axis at various locations along the longitudinal axis (e.g., in between each electrode (or a majority of the electrodes), in the middle of each electrode (or a majority of the electrodes) etc.) have generally the same cross-sectional area and shape), in an alternate embodiment, the teachings detailed herein can be applicable to a so-called tapered electrode, where the cross-sectional areas on planes taken normal to the longitudinal axis decrease with location towards the distal end of the electrode array.

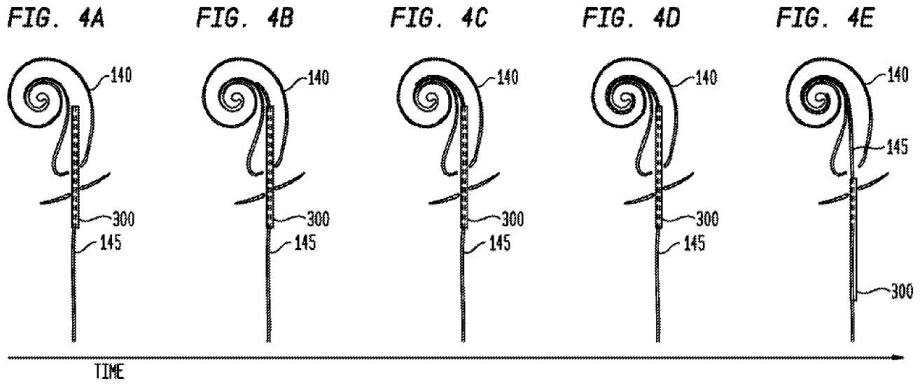

FIGS. 4A-4E depict an exemplary insertion regime of an electrode assembly according to an exemplary embodiment. As shown in FIG. 4A, the combined arrangement of an insertion guide tube 300 and electrode assembly 145 is substantially straight. This is due in part to the rigidity of insertion guide tube 300 relative to the bias force applied to the interior wall of the guide tube by pre-curved electrode assembly 145.

As noted, in some embodiments, the electrode assembly 145 is biased to curl and will do so in the absence of forces applied thereto to maintain the straightness. That is, electrode assembly 145 has a memory that causes it to adopt a curved configuration in the absence of external forces. As a result, when electrode assembly 145 is retained in a straight orientation in guide tube 300, the guide tube prevents the electrode assembly from returning to its pre-curved configuration. In the embodiment configured to be implanted in scala tympani of the cochlea, electrode assembly 145 is pre-curved to have a radius of curvature that approximates and/or is less than the curvature of medial side of the scala tympani of the cochlea. Such embodiments of the electrode assembly are referred to as a perimodiolar electrode assembly, and this position within cochlea 140 is commonly referred to as the perimodiolar position. In some embodiments, placing electrode contacts in the perimodiolar position provides utility with respect to the specificity of electrical stimulation, and can reduce the requisite current levels thereby reducing power consumption.

As shown in FIGS. 4B-4D, electrode assembly 145 may be continually advanced through insertion guide tube 300 while the insertion sheath is maintained in a substantially stationary position. This causes the distal end of electrode assembly 145 to extend from the distal end of insertion guide tube 300. As it does so, the illustrative embodiment of electrode assembly 145 bends or curves to attain a perimodiolar position, as shown in FIGS. 4B-4D, owing to its bias (memory) to curve. Once electrode assembly 145 is located at the desired depth in the scala tympani, insertion guide tube 300 is removed from cochlea 140 while electrode assembly 145 is maintained in a stationary position. This is illustrated in FIG. 4E.

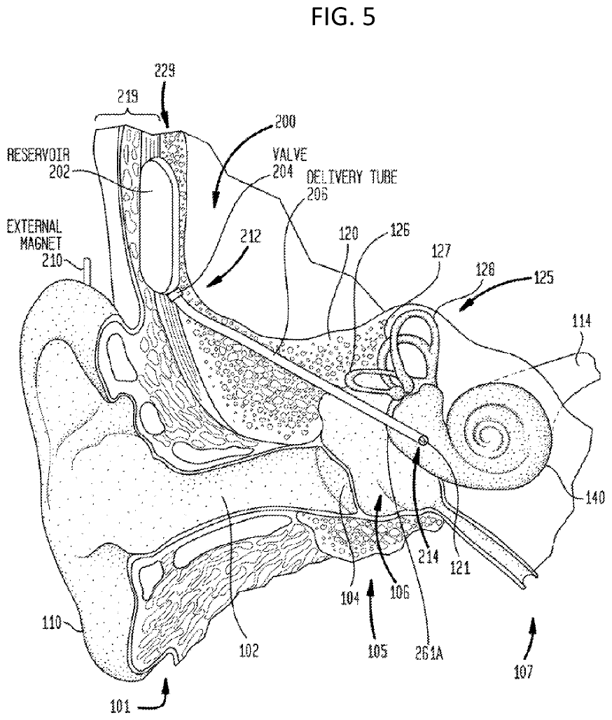

FIG. 5 depicts an exemplary drug delivery device, the details of which will be provided below. It can be utilitarian to have a prompt and/or extended delivery solution for use in the delivery of treatment substances to a target location of a recipient. In general, extended treatment substance delivery refers to the delivery of treatment substances over a period of time (e.g., continuously, periodically, etc.). The extended delivery may be activated during or after surgery and can be extended as long as is needed. The period of time may not immediately follow the initial implantation of the auditory prosthesis. Embodiments of the teachings herein can facilitate extended delivery of treatment substances, as well as facilitating prompt delivery of such substances.

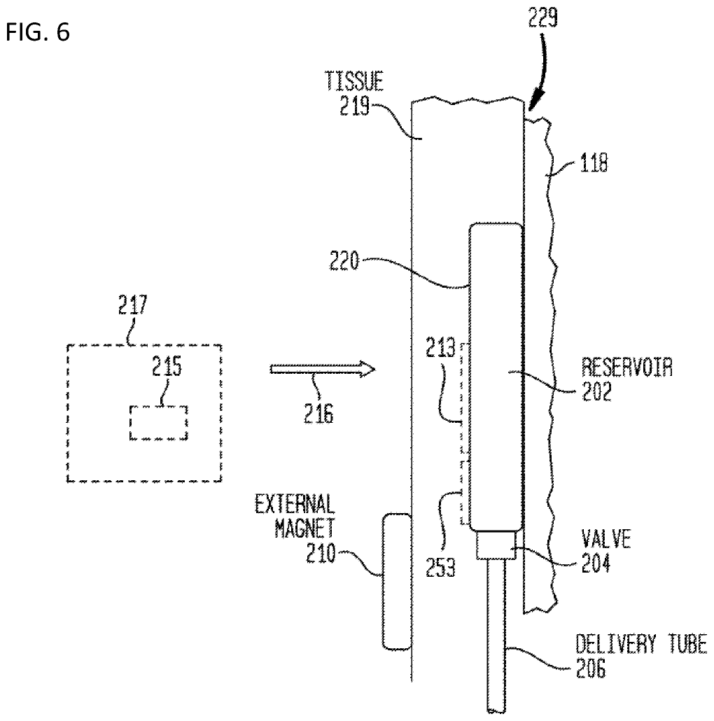

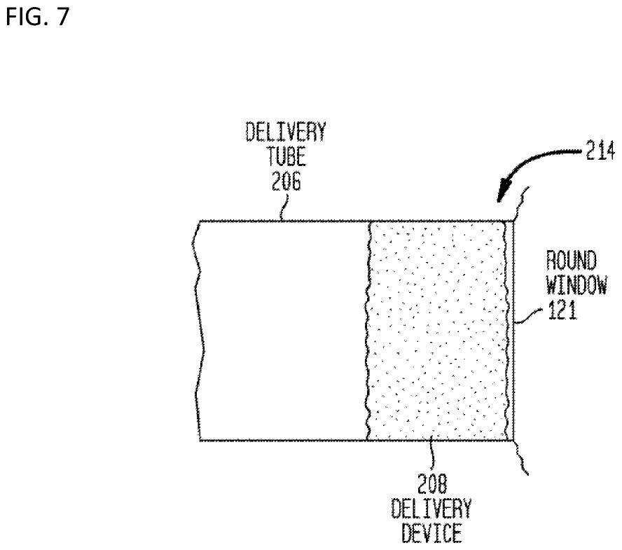

FIG. 5 illustrates an implantable delivery system 200 that can be utilized with the teachings detailed herein, and otherwise modified as detailed by way of example below. The delivery system has a passive actuation mechanism. However, it is noted that the delivery system 200 can also or instead have an active actuation system. The delivery system 200 is sometimes referred to herein as an inner ear delivery system because it is configured to deliver treatment substances to the recipient's inner ear (e.g., the target location is the interior of the recipient's cochlea 140). FIG. 6 illustrates a first portion of the delivery system 200, while FIG. 7 is a cross-sectional view of a second portion of the delivery system 200.

Delivery system 200 of FIGS. 5-7 comprises a reservoir 202, a valve 204, a delivery tube 206, and a delivery device 208 (FIG. 7). For ease of illustration, the delivery system 200 is shown separate from any implantable auditory prostheses. However, it is to be appreciated that the delivery system 200, and any of the other delivery systems detailed herein and/or variations thereof, could be used with, for example, cochlear implants, such as that presented in FIG. 1, direct acoustic stimulators, middle ear implants, bone conduction devices, etc. The implantable components (e.g., reservoir, valve, delivery tube, etc.) of delivery system 200 (or any other delivery system detailed herein) could be separate from or integrated with the other components of the implantable auditory prosthesis. Additionally, the delivery system 200 can include, or operate with, an external magnet 210, which is separate from or part of the implantable auditory prostheses, for purposes of, e.g., controlling operation of valve 204.

The reservoir 202 is positioned within the recipient underneath a portion of the recipient's skin/muscle/fat, collectively referred to herein as tissue 219. The reservoir 202 may be positioned between layers of the recipient's tissue 219 or may be adjacent to a subcutaneous outer surface 229 of the recipient's skull. For example, the reservoir 202 may be positioned in a surgically created pocket at the outer surface 229 (i.e., adjacent to a superior portion 118 of the temporal bone 115).

The reservoir 202 is, prior to or after implantation, at least partially filled with a treatment substance for delivery to the inner ear 107 of the recipient. The treatment substance may be, for example, in a liquid form, a gel form, and/or comprise nanoparticles or pellets. In certain arrangements, the treatment substance may initially be in a crystalline/solid form that is subsequently dissolved. For example, a reservoir could include two chambers, one that comprises a fluid (e.g., artificial perilymph or saline) and one that comprises the crystalline/solid treatment substance. The fluid may be mixed with the crystalline/solid treatment substance to form a fluid or gel treatment substance that may be subsequently delivered to the recipient.

The reservoir 202 includes a needle port (not shown) so that the reservoir 202 can be refilled via a needle injection through the skin. The reservoir 202 may be explanted and replaced with another reservoir that is, prior to or after implantation, at least partially filled with a treatment substance. The reservoir 202 may have a preformed shape and the reservoir is implanted in this shape. The reservoir 202 may have a first shape that facilitates implantation and a second shape for use in delivering treatment substances to the recipient. For example, the reservoir 202 may have a rolled or substantially flat initial shape that facilitates implantation. The reservoir 202 may then be configured to expand after implantation. Such may be used, for example, to insert the reservoir through a tympanostomy into the middle ear or ear canal, through an opening in the inner ear, or to facilitate other minimally invasive insertions. Reservoir 202 may have other shapes as needed to operate with hearing prostheses, as will be detailed below by way of example and not by way of limitation.

The delivery tube 206 includes a proximal end 212 and a distal end 214. The proximal end 212 of the delivery tube 206 is fluidically coupled to the reservoir 202 via the valve 204. As shown in FIG. 7, the distal end 214 of the delivery tube 206 is fluidically coupled to the recipient's round window 121. A delivery device 208 disposed within the distal end 214 of the delivery tube 206 is positioned abutting the round window 121. As described further below, the delivery tube 206 may be secured within the recipient so that the distal end 214 remains located adjacent to the round window 121.

FIGS. 5-7 illustrate a system that utilizes utilize a passive actuation mechanism to produce a pumping action to transfer a treatment substance from the reservoir 202 to the delivery device 208 at the distal end 214 of the delivery tube 206. More specifically, in this system, the reservoir 202 is compressible in response to an external force 216. That is, at least one part or portion of the reservoir 202, such as wall 220 or a portion thereof, is formed from a resiliently flexible material that is configured to deform in response to application of the external force 216. In some implementations of the system of FIG. 5, positioning of the reservoir 202 adjacent the superior portion of the mastoid provides a surface that is sufficiently rigid to counter the external force 216. As a result, a pressure change occurs in the reservoir 202 so as to propel (push) a portion of the treatment substance out of the reservoir through valve 204.

FIGS. 5 and 6 illustrate a specific arrangement in which the reservoir 202 includes a resiliently flexible wall 220. It is to be appreciated that the reservoir 202 can be formed from various resiliently flexible parts and rigid parts. It is also to be appreciated that the reservoir 202 may have a variety of shapes and sizes (e.g., cylindrical, square, rectangular, etc.) or other configurations. For example, the reservoir 202 could further include a spring mounted base that maintains a pressure in the reservoir 202 until the reservoir is substantially empty. Other mechanisms for maintaining a pressure in the reservoir may be used in other arrangements.

External force is applied on the tissue 219 adjacent to the reservoir 202 to create the external force. As will be described below, in some embodiments, an external vibratory device of a passive transcutaneous bone conduction device that vibrates to evoke a hearing percept is pressed onto the soft tissue 219 under which the reservoir 202 is located. The movement (e.g., oscillation/vibration) of the actuator causes deformations the reservoir 202 to create the pumping action that propels the treatment substance out of the reservoir.

Internal and/or external magnets and/or magnetic materials may be used in the arrangements of FIGS. 5 and 6 to ensure that the actuator 217 applies force at an optimal location of the reservoir 202. For example, the reservoir 202 may include a magnetic positioning member 213 located at or near an optimal location for application of an external force from the actuator 217. The actuator 217 may include a magnet 215 configured to magnetically mate with the magnetic positioning member 213. As such, when actuator 217 is properly positioned, the magnet 215 will mate with the magnetic positioning member 213 and the force from the actuator 217 will be applied at the optimal location.

A remote control, remotely placed actuator (subcutaneous or otherwise) may be alternatively used. For example, in a further arrangement, the implant includes implanted electronics 253 (shown using dotted lines in FIG. 6). These implanted electronics 253 may be configured to, for example, control the valve 204 and/or include an actuation mechanism that can force treatment substance from the reservoir 202. The implanted electronics 253 may be powered and/or controlled through a transcutaneous link (e.g., RF link). As such, the implanted electronics 253 may include or be electrically connected to an RF coil, receiver/transceiver unit, etc.

The implanted electronics 253 may include or be connected to a sensor that is used, at least in part, to assist in control of delivery of the treatment substance to the recipient. For example, a sensor (e.g., a temperature sensor, a sensor to detect infection or bacteria growth, etc.) may provide indications of when a treatment substance should be delivered and/or when delivery should be ceased for a period of time. A sensor may also be configured to determine an impact of the treatment substance on the recipient (e.g., evaluate effectiveness of the treatment substance).

As noted, the treatment substance (sometimes herein referred to as therapeutic substance) is released from the reservoir 202 through the valve 204. The valve 204 may be a check valve (one-way valve) that allows the treatment substance to pass therethrough in one direction only. This assures that released treatment substances do not back-flow into the reservoir 202. The valve 204 is a valve that is configured to open in response to the pressure change in the reservoir 202 (e.g., a ball check valve, diaphragm check valve, swing check valve or tilting disc check valve, etc.). The valve 204 may be a stop-check valve that includes an override control to stop flow regardless of flow direction or pressure. That is, in addition to closing in response to backflow or insufficient forward pressure (as in a normal check valve), a stop-check value can also be deliberately opened or shut by an external mechanism, thereby preventing any flow regardless of forward pressure. The valve 204 may be a stop-check value that is controlled by an external electric or magnetic field generated by, for example, the external magnet 210, an electromagnet, etc. In the system of FIGS. 5 and 6, the valve is responsive to a magnetic field generated by external magnet 210. As such, the valve 204 will open when the external magnet 210 is positioned in proximity to the valve 204 and will close when the external magnet 210 is removed from the proximity of the valve 204. Variable magnet strengths of external magnets may be used to control the dosage of the treatment substance. Additionally, an electromagnet may be used in place of the external magnet 210.

The use of a stop-check valve can prevent unintended dosing of the treatment substance when, for example, an accidental external force acts on the reservoir 202. The reservoir 202 is formed such that an increase in pressure of the reservoir 202 without an accompanying treatment substance release will not damage (i.e., rupture) the reservoir.

The use of a magnetically activated stop-check valve is merely exemplary and that other types of valves may be used. For example, the valve 204 may be actuated (i.e., opened) in response to an electrical signal (e.g., piezoelectric valve). The electrical signal may be received from a portion of an auditory prosthesis (not shown) that is implanted with the delivery system 200 or the electrical signal may be received from an external device (e.g., an RF actuation signal received from an external sound processor, remote control, etc.). In some instances, manually applied (e.g., finger) force be also able to open the valve 204.

Once the treatment substance is released through valve 204, the treatment substance flows through the delivery tube 206 to the delivery device 208. The delivery device 208 operates as a transfer mechanism to transfer the treatment substance from the delivery tube 206 to the round window 121. The treatment substance may then enter the cochlea 140 through the round window 121 (e.g., via osmosis). The delivery device 208 may be, for example, a wick, a sponge, permeating gel (e.g., hydrogel), etc.

The reservoir 202 may include a notification mechanism that transmits a signal or notification indicating that the reservoir 202 is substantially empty and/or needs refilled. For example, one or more electrode contacts (not shown) may be present and become electrically connected when the reservoir is substantially empty. Electronic components associated with or connected to the reservoir 202 may accordingly transmit a signal indicating that reservoir needs filled or replaced.

FIGS. 5-7 illustrate a specific example in which the round window 121 is the target location. As noted above, the round window 121 is an exemplary target location and other target locations are possible. FIGS. 5-7 also illustrate that the reservoir 202 is positioned adjacent to the outer surface 229 of the recipient's skull so that an external force may be used to propel the treatment substance from the reservoir.

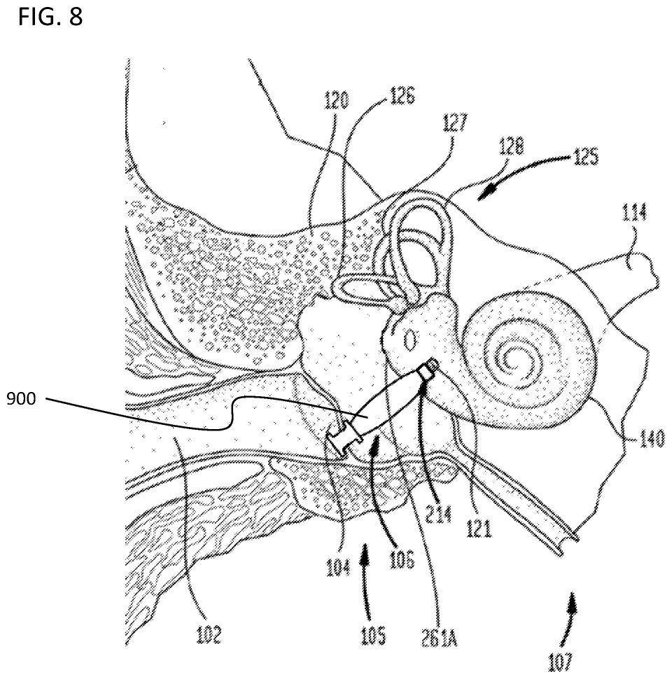

FIG. 8 presents an exemplary embodiment that is different than the embodiment of FIGS. 5-7. In this exemplary embodiment, there is a therapeutic substance delivery device 900, which is essentially located entirely in the middle ear cavity 106 (in this embodiment, a grommet extends through the tympanic membrane 104—more on this below). In this embodiment, the device 900 extends from the tympanic membrane 104 to the wall 261A of the cochlea that is exposed to the middle ear cavity 106. Therapeutic substance distribution and 214 of the therapeutic substance delivery device 900 can be attached to a round window niche, as will be described in greater detail below.

It is briefly noted that while the embodiment of FIG. 8 presents a middle ear cavity 106 without ossicles, in an alternative embodiment, the ossicles are present and/or otherwise functional. This will be described in greater detail below. It is also noted that while the embodiment of FIG. 8 depicts the utilizations of the teachings detailed herein in the absence of another prosthesis, such as a cochlear implant or a middle ear implant, it is to be noted that any disclosure herein of any embodiment associated with the therapeutic substance delivery device corresponds to a disclosure of the utilization of such with any of the other prostheses detailed herein unless otherwise noted.

Further, it is noted that while some embodiments of the teachings detailed herein are utilized to treat the effects associated with implanting a component in the ear system of the recipient, such as by way of example only and not by way of limitation, providing anti-inflammatory substances and/or steroids to the cochlea following a cochlear implant electrode array insertion, other embodiments of the teachings detailed herein are not utilized per se with an implant. In this regard, the teachings detailed herein can be utilized to treat hearing problems irrespective of whether or not the recipient utilizing the prosthesis. By way of example only and not by way of limitation, in an exemplary embodiment, the teachings detailed herein can be utilized to treat a syndrome that is attacking the hair cells of the cochlea prior to the utilization of a hearing prosthesis—even in some instances—by the recipient. That said, the teachings detailed herein can be utilized in isolation from any other prostheses. It is also noted that the teachings detailed herein can be used in combination with conventional hearing aids. In this regard, the teachings detailed herein can be utilized to treat ailments associated with the hearing and/or balance system of a recipient that may or may not rise to the level of requiring an implantable and/or partially implantable hearing prosthesis.

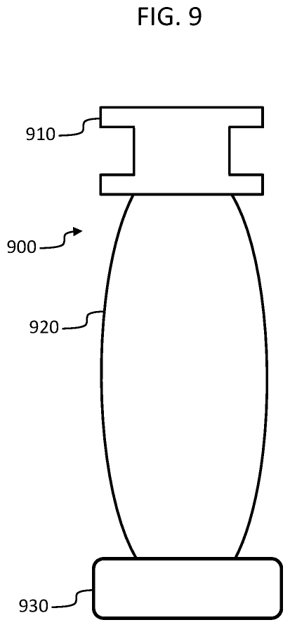

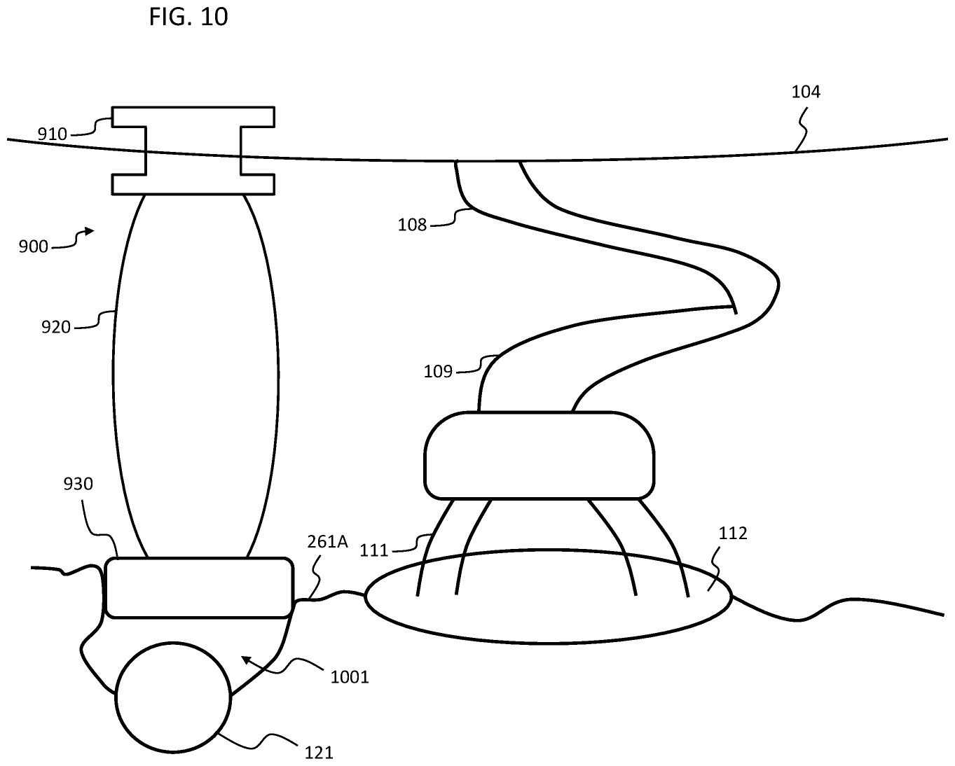

FIG. 9 presents a side view of the therapeutic substance delivery device 900. FIG. 10 presents an exemplary conceptual side view of the therapeutic substance delivery device 900 implanted in a middle ear cavity with a fully functioning ossicles (note that in reality, the round window 121 would likely be more parallel to the bottom of element 930 than that shown—FIG. 10 presents the window as round for description (and the oval window as oval) for description purposes—both might look like flat lines from the side). In this exemplary embodiment, there is grommet 910, reservoir 920, and substance transfer component 930. These elements will be described in order.

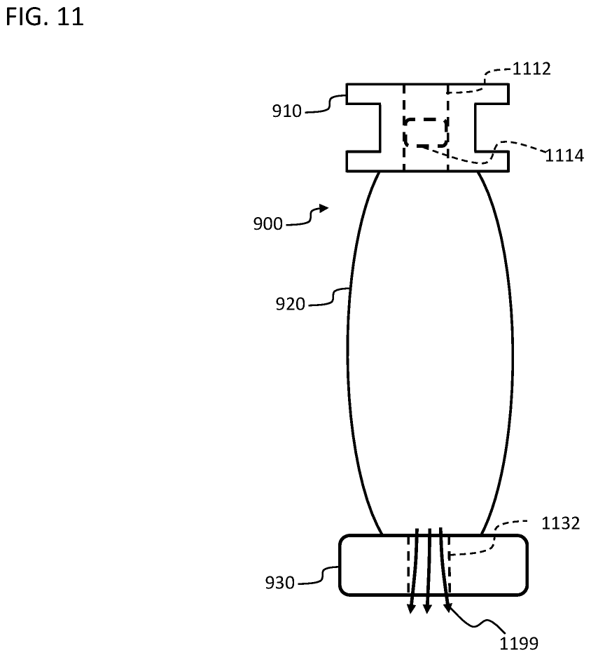

Grommet 910 can be a grommet as utilized for pressure relief/pressure equalization tubes or any other device that will permit access from outside the middle ear on one side of the tympanic membrane to the other side of the tympanic membrane in the middle ear. FIG. 11 depicts hidden lines representing the passageway 1112 through the grommet to the reservoir 920. Interposed inside the grommet is septum 1114. In an exemplary embodiment, a needle or lumen can be utilized to pierce the septum 1114 so that the reservoir 920 can be initially filled after implantation and/or refilled after the initial charge of therapeutic substance is depleted. In an exemplary embodiment, septum 1114 is a septum analogous to or otherwise the same as that which would be on a vaccine or drug container that enables a needle to access the interior of the container so that drug can be withdrawn and then the septum closes upon the withdrawal of the needle.

In some embodiments, the implantable apparatus has a refill system based on a middle ear pressure equalization tube.

In an exemplary embodiment, the tympanic membrane is pierced and the grommet 910 is placed in the piercing to provide an essentially permanent passageway through the tympanic membrane, while maintaining the structural integrity and the functionality of the tympanic membrane in at least some exemplary embodiments. As will be described in greater detail below, this piercing through the tympanic membrane is the route through which the entire therapeutic substance delivery device 900 is inserted into the middle ear cavity in an exemplary embodiment. However, for the moment, an elements by elements approach with respect to the description of the embodiment of FIG. 9 is first undertaken.

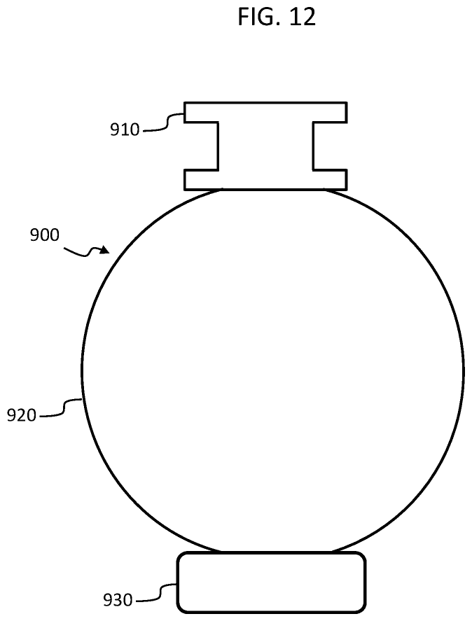

In fluid communication with the grommet, or, more accurately, a passageway through the grommet, is reservoir 920. In an exemplary embodiment, reservoir 920 is a flexible balloon or an analogous device or otherwise a flexible bag or bladder, etc., that is collapsible (and thus has utilitarian value with respect to transferring the device to the piercing through the tympanic membrane). In an exemplary embodiment, the body of the reservoir is made of an elastomeric material, such as an elastomeric membrane or an elastomeric sheet that has as its relaxed state a contracted state, as seen, for example, in FIG. 9, which can expand under the pressure of therapeutic substance delivered through the grommet 910 into the reservoir. In an exemplary embodiment, the therapeutic substance expands the reservoir outward, as shown for example in FIG. 12, and the tensile force on the wall of the reservoir seeks to contract the reservoir and thus applies a pressure on the therapeutic substance therein, thus encouraging the therapeutic substance to be transferred out of the reservoir.

The therapeutic substance transfer device 930 is located at the base of the device 900. In an exemplary embodiment, this transfer device 930 can be considered an “applicator foot.” In an exemplary embodiment, as is represented by way of example only and not by way of limitation with respect to FIG. 11, this can be a component that has a passageway 1132 that extends from the reservoir side to the opposite side. In an exemplary embodiment, the therapeutic substance transfer device 930 is a flexible puck or a similarly shaped body (round outer circumference with flat top and bottom, for example) or an oblong shaped cross-sectional device with a passageway therethrough. The flexible nature of the puck permits the puck to be interference fitted into the round window niche 1001 and thus held in place due to the interference fit. The puck can be sized and dimensioned to be located proximate the round window or in a prepared recess in the bone surrounding the cochlea. The therapeutic substance can travel through the passageway through the puck (as represented by the arrows 1199) and thus fill or at least “empty” into the void between the puck and the round window in the niche. The therapeutic substance can then leech or otherwise transfer, including active transfer, through the round window into the cochlea. In this regard, FIG. 10 presents an exemplary embodiment of a refillable therapeutic substance delivery device securable to a round window niche of a recipient, wherein the therapeutic substance delivery device is refillable while the therapeutic substance delivery device is secured to the round window niche.

In an exemplary embodiment, the transfer device 930 can include a flow restrictor. In an exemplary embodiment, the transfer device 930 is configured to meet or otherwise control the amount of therapeutic substance that flows from the reservoir therethrough. In some embodiments, the material and/or the structure thereof establishes the restriction. In other embodiments, it can be an active valve or a passive valve or the like that establishes the restriction of the flow out of the reservoir. Any device, system, and/or method that can restrict flow can be utilized in at least some exemplary embodiments.

Accordingly, in an exemplary embodiment, the therapeutic substance delivery device 930 or the variations thereof disclosed herein, or any other alternative embodiment is configured to deliver therapeutic substance from the reservoir into a cochlea of the recipient across a round window membrane. In an exemplary embodiment, this is done via diffusion across the round window membrane. As will be described in greater detail below, in an alternate embodiment, there is actual piercing through the round window membrane. Further, as will be described in greater detail below, in an alternate embodiment, the therapeutic substance is delivered instead via the oval window membrane and/or via an anatomical structure attached thereto. In still further embodiments, the therapeutic substance is delivered to the cochlea via a cochleostomy away from the windows, or via a drilled bony recess that does not open the cochlea but exposes periosteum, or simply reduces the amount of tissue between the applicator and the fluid filled chambers of the cochlea.

In an exemplary embodiment, the therapeutic substance delivery device is configured to transfer therapeutic substance into the cochlea via diffuse osmosis.

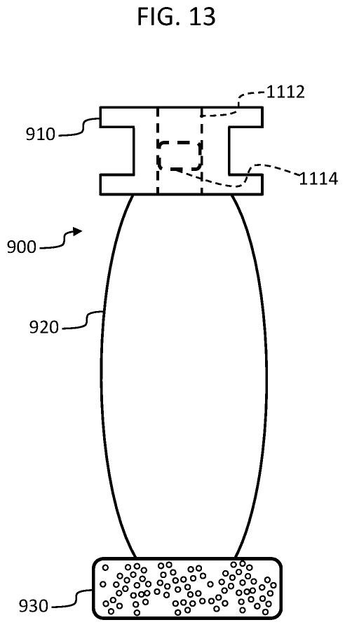

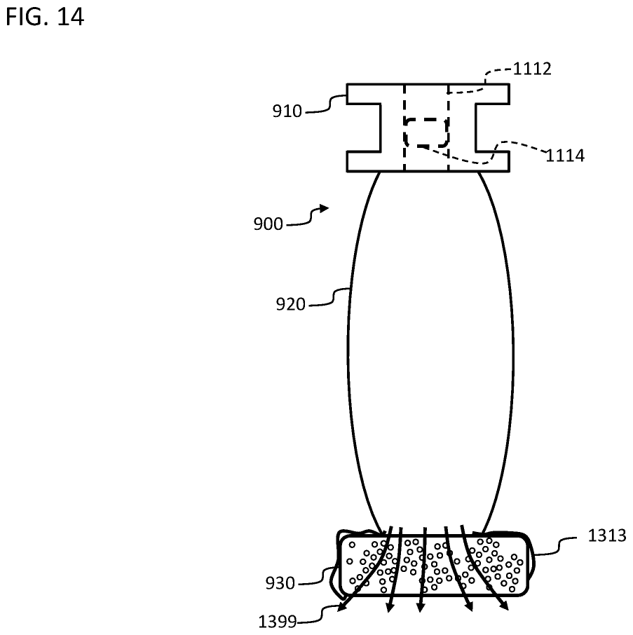

The above said, FIG. 13 presents an alternate embodiment where the therapeutic substance transfer device 930 is a porous sponge body. This can fit into the round window niche in a manner analogous to or otherwise the same as the above detailed puck. In an exemplary embodiment, owing to the much more flexible nature of the sponge material of this embodiment, in some embodiments, substance transfer device 930 is placed directly against the round window, and the therapeutic substance flows from the sponge directly to the membrane of the round window and then enters the cochlea therethrough. Because the sponge is very flexible, it does not significantly or otherwise effectively impede movements of the round window. In an exemplary embodiment, the sponge absorbs, soaks up and/or mops up the therapeutic substance from the reservoir (in an embodiment, the sponge/puck establishes one of the barriers of the reservoir—in some other embodiments, there is a valve or a metering device in between the reservoir and the therapeutic substance transfer device 930). In some embodiments, a wicking action and or a capillary action are executed by the puck/sponge or other device to move the therapeutic substance. In this regard, in an exemplary embodiment, the sponge can be utilized in an arrangement where the reservoir is not pressurized or otherwise does not establish a pressure. The sponge can also be utilized in a pressurized system. FIG. 13 depicts arrows 1390 representing the travel of therapeutic substance from the reservoir 920 through the porous body/sponge of 930. As can be seen, the arrows travel in a more diffuse pattern than that which was the case with respect to the passageway through the transfer device 930. In an exemplary embodiment, the walls of the round window niche can guide the therapeutic substance or otherwise corral the therapeutic substance towards the round window. Alternatively, and/or in addition to this, a barrier 1313 can extend about the side and/or the top of the therapeutic substance transfer device 930 to prevent the therapeutic substance from traveling in a direction other than downward/towards the round window. In this regard, barrier 1313 is presented as a flexible polymer wall that has an opening that opens up into the reservoir and which extends from the reservoir around the sides of the therapeutic substance transfer device 930. This can be considered an inverted thin-walled cup or saucer with a hole in the bottom (top in the inverted state). As seen in this embodiment, the barrier 1313 can extend completely from one side of the transfer device 930 to the other side, as is the case on the left side, and/or extend from one side towards the other side but not all the way thereto. Any arrangement that can enable the teachings detailed herein can be utilized in at least some exemplary embodiments.

Thus, in view of the above, there is an implantable apparatus that includes a reservoir and a sponge and/or a porous body in fluid communication with the reservoir. In an exemplary embodiment, the apparatus is configured such that the sponge and/or porous body is in direct contact with a window of a cochlea and/or an anatomical structure that is attached to the window when the device is implanted in a recipient so that therapeutic substance in the reservoir can travel through the sponge and/or porous body to the window.

In an exemplary embodiment, device 930 is any eluting component that elutes therapeutic substance therefrom.

In an exemplary embodiment, element 930 is a silicone body and/or polymer membrane and/or expanded PTFE body in fluid communication with the reservoir. In an exemplary embodiment, the delivery device is configured such that the silicone body and/or polymer membrane and/or expanded PTFE body is in direct contact with a window of a cochlea and/or an anatomical structure attached to the window when the device is implanted in a recipient so that therapeutic substance in the reservoir can travel through the silicone body and/or polymer membrane and/or expanded PTFE body to the window.

In an exemplary embodiment, the therapeutic substance transfer device 930 will be made entirely or partially out of a polymer material. This can also be the case with respect to the reservoir and/or the grommet. Indeed, the entire therapeutic substance delivery device can be made out of such.

The therapeutic substance transfer device can be made out of silicone, it can be a polymer membrane. The therapeutic substance transfer device can be a device that transfers the therapeutic substance in a spongelike manner and/or a wick type application. The transfer device can be a fibrous component, it can be a rubber like component and/or can be a spongelike component.

Some additional variations of the therapeutic substance transfer device 930 will be described in greater detail below. It is briefly noted that any device, system, and/or method that can enable therapeutic substance transfer from the reservoir to the round window (or oval window or anatomic structure attached thereto in some other embodiments—more on this below) can be utilized in at least some exemplary embodiments unless otherwise noted.

In view of the above, it can be seen that in an exemplary embodiment, there is an apparatus comprising a refillable therapeutic substance delivery device including a reservoir, the reservoir being configured to be located in an adult middle ear cavity of a human recipient and/or a pre-adult and/or an adolescent and/or a pre-adolescent middle ear cavity of a human recipient or of any given human. In accordance with the teachings above, in an exemplary embodiment, the entire reservoir is located in the middle ear cavity. Still further, as seen above, in at least some exemplary embodiments, the therapeutic substance delivery device is configured such that the reservoir is accessible through a tympanic membrane of the recipient. The embodiments above depict the utilization of the grommet 910. In this regard, with respect to the embodiment of FIG. 10, for example, the therapeutic substance delivery device is configured for implantation into a middle ear of a recipient and to always be in contact with a tympanic membrane of the recipient other than a permanent explantation of the device. In an exemplary embodiment, the substance delivery device is configured to be in contact with a tympanic membrane for at least 3, 4, 5, 6, 7, 8, 9, 10, 11, 12, 13, 14, 15, 16, 17, 18, 19, 20, 21, 22, 23, 24, or 25 months, or years, or any value or range of values therebetween in at least integer increments (4 to 14 months, 7 to 11 years, etc.). That said, in an exemplary embodiment, the delivery device is configured for implantation into a middle ear of the recipient and to never be in contact with the tympanic membrane, at least after the initial implantation (again, some embodiments entail inserting the delivery device through the puncture in the tympanic membrane, which may involve contact during implantation—more on this below).

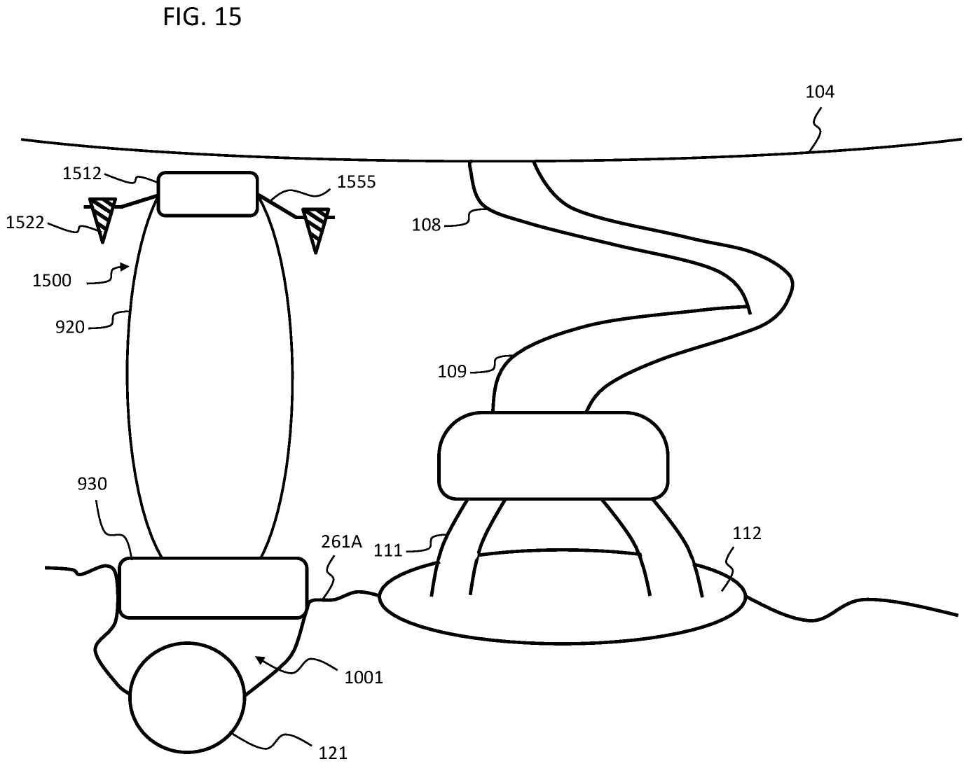

Along the lines articulated above, in an alternate embodiment, as seen in FIG. 15, there is no component that extends outside the middle ear. More particularly, in the embodiment of the therapeutic substance delivery device 1500 presented in FIG. 15, the grommet is not present (or in an alternative embodiment, it is present, but it is located in board of the tympanic membrane 104). Instead, a septum 1512 is located in board of the tympanic membrane, and supported by brackets 1555 that are attached to the wall of the middle ear by bone screws 1522. (In an alternative embodiment, instead of bone screws, clips or clamps or the like can be utilized that attached to promontory structures within the middle ear cavity. Any arrangement that can support the septum and/or the reservoir 920 and/or the delivery device that can enable the teachings detailed herein can be utilized in at least some exemplary embodiments.) The brackets 1555 hold the septum in place, and also hold the rest of the therapeutic delivery device in place, or at least the proximal end thereof. In this exemplary embodiment, to fill or refill the reservoir 920, a needle or lumen is utilized to pierce the tympanic membrane 104 and then to pierce the septum 1512 so that the therapeutic substance can be transferred from outside the middle ear cavity into the reservoir 920 which is located inside the middle ear cavity. In an exemplary embodiment, the piercing can be of sufficient low trauma that the tympanic membrane heals and also permits the re-piercing of the tympanic membrane at a later date for refilling/recharging of the reservoir 920.

The above said, in an exemplary embodiment, a grommet can also be located on the tympanic membrane 104, which grommet would not be in contact with the septum with the reservoir or any other part of the therapeutic substance delivery device. Indeed, in an exemplary embodiment, the grommet would not be part of the delivery device, but would instead be an access component that enables access to the therapeutic substance delivery device. In an exemplary embodiment, the lumen can be placed to the grommet and then into and through the septum 1512, which is held in place and the same or a similar manner as depicted in FIG. 15.

It is also noted that while the embodiment shown above has depicted a grommet that extends the tympanic membrane, in an alternate embodiment, a flange or the like can be utilized, which flange is only located on one side of the tympanic membrane. In an exemplary embodiment, the flange can be stapled or glued or otherwise adhered to the tympanic membrane.

Any device, system, and/or method that can enable securement of at least one end of the therapeutic substance delivery device to the tympanic membrane and/or any device, system, and/or method that can provide an injection port so that the reservoirs can be refilled can be utilized in at least some exemplary embodiments.

Note further that in an exemplary embodiment, the grommet can also have a septum therein. This can establish a closure of the middle ear cavity when the lumen/needle is not extending through the grommet. Thus, in at least some exemplary embodiments, there is a therapeutic substance delivery device that includes a grommet attachable to a tympanic membrane through which the reservoir can be accessed to refill the reservoir. In other embodiments, the delivery device does not include a grommet, at least not one that is part of the delivery device per se.

In some embodiments, adhesives can be utilized to hold the working end and/or the refilling and of the therapeutic substance delivery device in place. In an exemplary embodiment, the proximal end can be adhesively attached to the tympanic membrane (or to the wall of the middle ear or to an anatomical structure thereof) and/or the distal end can be adhesively attached to the wall of the middle ear/portion of the cochlea that establishes a boundary of the middle ear. In an exemplary embodiment, the distal end can be adhesively attached to the round window niche. It is noted that a combination of adhesive interference fitting can be utilized in some embodiments. In an exemplary embodiment, surface tension can be utilized to maintain the applicator foot at the given location. Further, in an exemplary embodiment, there might even be noncontact between the applicator foot and the wall of the middle ear cavity and/or the anatomical structure associated there with. In an exemplary embodiment, the therapeutic substance delivery device can be mounted in the middle ear cavity such that the applicator foot (actually, it would no longer be a foot as much as it would be an applicator platform or nozzle or therapeutic substance exit) is maintained in space proximate but away from contact with the tissue of the recipient. In an exemplary embodiment, a cage or bracket structure or the like can extend away from various sides and/or the face of the therapeutic substance transfer device 930, which components interface with an otherwise contact the wall of the middle ear cavity.

In an exemplary embodiment, a device can be utilized to physically lock the distal end of the therapeutic substance delivery device to the promontory (any of the teachings detailed herein can be utilized to interface with the promontory) and/or the round window niche. In an exemplary embodiment, a spring-loaded apparatus can spring out underneath the bony structure to lock the device at that location. Alternatively, and/or in addition to this, a mechanically actuated device can be utilized, such as a jackscrew device or the like. In an exemplary embodiment, an aspect ratio of a component can change, such as changing an “0” shaped component to an oval shaped component. The total outside circumference of the component can be the same, but the length will extend and the width will contract, the length being utilized as the component that locks the device in the niche. In an exemplary embodiment, a component can go into the niche in one orientation and then change like an articulating anchor of the like, and thus “trap” the distal end of the device in the niche.

In an exemplary embodiment, the distal end can include a component that inflates or otherwise expands once the component is located in the round window niche so as to secure the distal end at that location. An inflatable balloon or an expanding material that expands once exposed to a physical phenomenon (UV light, sound, electricity, etc.) or something that is constrained and then the constraint is removed so that the component expands can be utilized in at least some exemplary embodiments.

In an exemplary embodiment, a gel or some other substance that can enable the transfer of therapeutic substance from the device to the round window can be utilized. In an exemplary embodiment, gel can be placed into the round window niche/the round window niche can actually be filled with gel, and the applicator end of the delivery device can interface with the gel. The device could transfer the therapeutic substance into the gel and the gel would conduct the therapeutic substance to the round window.

In an exemplary embodiment, the therapeutic substance delivery device can be self-supporting with respect to the attachment at the round window niche or whatever component to which the distal ends attached and/or could also be attached to a second location in the middle ear (any location that can enable such can be utilized at least some exemplary embodiments).

In an exemplary embodiment, the therapeutic substance transfer device or otherwise the applicator foot can be a component that expands once located at the fixation position. For example, the therapeutic substance transfer device can be placed into the round window niche, and then stimulation can be applied thereto to cause the transfer device to expand or otherwise fill in the opening thereof. This can have utilitarian value both with respect to securing the distal end of the delivery device and also enclosing the area of delivery. Principles of operation can include the utilization of hydrostatic pressure and/or weak adhesion or strong adhesion to secure the distal end.

Again, in at least some exemplary embodiments, the round window niche is enclosed by the device and is utilized as a natural conduit to the round window. In an exemplary embodiment, instead of being located in the round window niche, the therapeutic substance delivery device surrounds the round window niche like a suction cup as noted herein.

Still further, in an exemplary embodiment, a suction cup arrangement or the like can be utilized to hold the distal end that the location proximate the round window and/or over window. Moreover, Velcro could be utilized. Any arrangement that can the distal end in place can be utilized in at least some exemplary embodiments. Moreover, any device, system, and/or method that can hold the applicator against the tissue of interest, such as the round window, the oval window, or the oval window footplate, can be utilized in at least some exemplary embodiments. Again, some exemplary embodiments of the therapeutic substance transfer device are configured for direct contact with the round window and/or oval window and/or oval window footplate, while other embodiments position that device away from those anatomical features.

Note also that the concept of the utilization of the flange 1555 and the bone screws can also be applied to the distal end of the therapeutic substance delivery device. In an exemplary embodiment, a flange is attached to the distal end of the reservoir 920 and/or to the therapeutic substance transfer device 930. In an exemplary embodiment, the flange can extend around the reservoir 920 and apply a downward force onto the therapeutic substance transfer device when the flange is connected to the outer wall of the cochlea. That said, in an alternative embodiment that avoids utilizing bone screws or the like against the outer wall of the cochlea, the flange can extend upwards or outwards towards portions of the middle ear that are away from the wall of the cochlea. Still further, in an exemplary embodiment, the flange can be adhesively attached to the wall of the middle ear and/or to an anatomical structure therein. This is the case with the flange of the proximal end and the flange of the distal end.

Note also that some embodiments include adhesively connecting portions of the therapeutic substance delivery device to artificial components that are secured to the walls of the middle ear and/or to anatomical structures therein. By way of example only and not by way of limitation, a bone screw or the like can be attached to the wall of the middle ear, where the intention is to not move that bone screw for the life of the recipient, and a component of the therapeutic substance delivery device can be adhesively adhered to the head of that bone screw and/or to a flange that is connected that bone screw, which adhesive connection is easier to “break” or otherwise undo than that which would correspond to removing the bone screw or otherwise presents less failure mode scenarios. For example, the adhesive could be an adhesive that is uncured or otherwise degrades in the presence of ultraviolet light or the like. Thus, months or years after the implantation, by exposing the adhesive to ultraviolet light, or to light of a certain wavelength, etc., the adhesive will come undone and the device can be explanted without removing the bone screws. Note also that the concept of a weakening adhesive can also be applied to embodiments where adhesive is applied between the device and the middle ear or other anatomic structures in the middle ear.

In any event, as can be seen, in an exemplary embodiment, there is a therapeutic substance delivery device that is configured such that the reservoir is accessible through a tympanic membrane of the recipient. In an exemplary embodiment, this access enables refilling of the therapeutic substance delivery device from outside the middle ear cavity. Thus, in at least some exemplary embodiments, there is a therapeutic substance delivery device that is attachable to a tympanic membrane while functioning ossicles are attached thereto, while in other embodiments, the delivery device is not attachable to a tympanic membrane or otherwise is not attached to the tympanic membrane. Also, in at least some exemplary embodiments, regardless of how the therapeutic substance delivery device is attached, the delivery device does not have a significant impact on the performance of the ossicles and/or on the movements of the tympanic membrane.

Thus, in at least some exemplary embodiments, the therapeutic substance delivery device is configured to extend from a location at least proximate a tympanic membrane to a round window niche of a cochlea. In an exemplary embodiment, the device extends all the way from the tympanic membrane to the round window niche. In an exemplary embodiment, the therapeutic substance delivery device extends from the tympanic membrane or a location proximate the tympanic membrane to the round window or to the oval window. In an exemplary embodiment, the therapeutic substance delivery device extends from the tympanic membrane or a location proximate to the tympanic membrane to an anatomical structure connected to the oval window. Further, in an exemplary embodiment, the therapeutic substance delivery device extends from the tympanic membrane or a location proximate to the tympanic membrane to a wall of the cochlea that establishes the middle ear cavity/to a cochleostomy.

As noted herein, in an exemplary embodiment, the therapeutic substance delivery device does not contact the round window and/or oval window and/or oval window footplate. Further, in an exemplary embodiment, the applicator or otherwise the location where the therapeutic substance leaves the therapeutic substance delivery device can be located reasonably far away from the target tissue. As will be detailed below, in an exemplary embodiment, the delivery device drips the therapeutic substance on to the tissue. Accordingly, in an exemplary embodiment, the therapeutic substance delivery device can be implanted and utilized such that no part of the delivery device is within 0.1, 0.2, 0.3, 0.4, 0.5, 0.6, 0.7, 0.8, 0.9, 1.0, 1.25, 1.5, 1.75, 2, 2.25, 2.5, 2.75, 3, 3.25, 3.5, 3.75, 4, 4.5, 5, 5.5, 6, 6.5, 7, 7.5, 8, 9, or 10 mm, or any value or range of values therebetween in 0.01 mm increments from the target tissue (e.g., round window, oval window, oval window footplate, etc.).

With regard to an anatomical structure that is connected to a window, such as the oval window, in an exemplary embodiment, the anatomical structure can be a stapes footplate. In this regard, the apparatus that delivers a therapeutic substance can interface with the stapes footplate when the apparatus is operationally implanted in the recipient. By operationally implanted, it is meant that the apparatus is actually functioning. In an exemplary embodiment, at least some of the therapeutic substance delivery systems detailed herein are configured to be operationally implanted in the recipient for the aforementioned temporal periods detailed above consistent with the embodiments where the delivery device is refillable, as no non-refillable therapeutic substance delivery device will continuously deliver therapeutic substance for the time frames associated with the teachings detailed herein, and thus will not be operationally implantable for such even though the device might still remain in the recipient beyond the time that it effectively stops delivering therapeutic substance to the recipient or otherwise completely stops delivering therapeutic substance to the recipient.

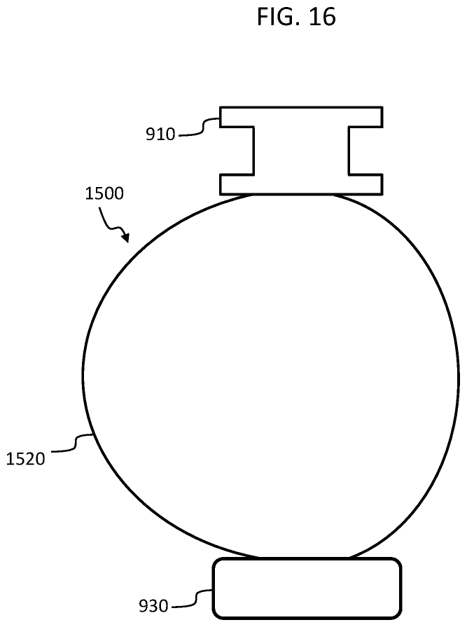

As seen in FIG. 10 above, at least some exemplary embodiments are utilized with a fully intact ossicles. Thus, there can be utilitarian value with respect to a therapeutic substance delivery device that avoids contact with the ossicles or otherwise does not interfere with their operation. In this regard, in an exemplary embodiment where the reservoir is an expandable reservoir, the reservoir can be configured to expand only on one side and/or otherwise expand in a more limited amount on the side that is facing the ossicles. FIG. 16 presents an exemplary embodiment of an exemplary therapeutic substance delivery device 1500 where the reservoir 1520 is configured to expand more on one side and the other. Actually, in an exemplary embodiment, the reservoir expands in three of the four directions away from the longitudinal axis of the deflated/unexpanded reservoir, and expands less or otherwise does not expand at all on the side that faces the ossicles. Thus, the therapeutic substance delivery device according to some embodiments can be placed relatively close to the ossicles or a component thereof, and also have the expansion feature without interfering with the ossicles or otherwise coming into contact with the ossicles.

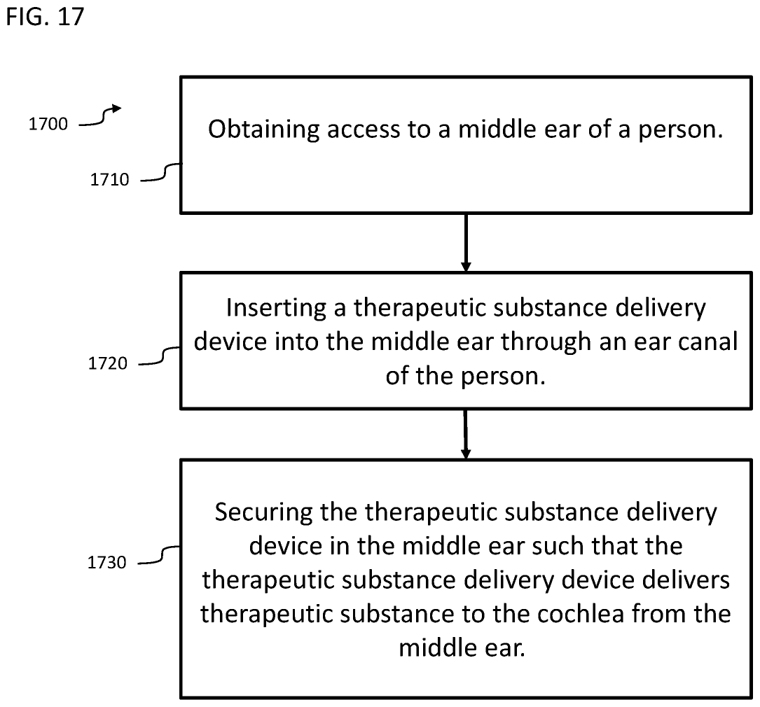

FIG. 17 presents an exemplary flowchart for an exemplary method, method 1700, according to an exemplary embodiment. Method 1700 includes method action 1710, which includes obtaining access to a middle ear of a person. In at least some exemplary embodiments, this can include executing a tympanostomy. In some embodiments, the tympanic membrane is completely removed, such as in scenarios where the recipient has completely lost all hearing, which can be the case with respect to a recipient that receives a cochlear implant. In some embodiments, the tympanic membrane is maintained in as pristine a state as possible. In an exemplary embodiment, a substantial portion of the tympanic membrane can be removed or otherwise pierced relative to that which would be the case for a tympanostomy. Any method or system or device that will enable access to the middle ear through the ear canal can be utilized in at least some exemplary embodiments.

In an exemplary embodiment, all access to the middle ear that occurs during the implantation surgery or process occurs through the ear canal. In an exemplary embodiment, no portion of the surgery includes accessing the middle ear through a route that is outside the ear canal. Note that this does not exclude accessing the middle ear for other reasons through other routes, such as, for example, that which results in the application of a cochlear implant electrode array or a middle ear actuator, etc. Indeed, in at least some exemplary embodiments, the teachings detailed herein are practiced to remediate or otherwise address scenarios that occur after implantation of a middle ear implant and/or a cochlear implant, etc. Thus, some embodiments specifically include accessing a middle ear cavity of a recipient according to the teachings detailed herein where such cavity was previously accessed to implant another type of device.

In an exemplary embodiment, the implantation process of the therapeutic substance delivery device begins 1, 2, 3, 4, 5, 6, 7, 8, 9, 10, 11, 12, 13, 14, 15, 20, 25, 30, 35, 40, 45, or 50 days, or weeks, or months, or years, or any range of values therebetween in integer increments after the middle ear cavity was accessed (however it was accessed) to implant a device other than the therapeutic substance delivery device.

Method 1700 further includes method action 1720, which includes inserting a therapeutic substance delivery device into the middle ear through an ear canal of the person. In an exemplary embodiment, a grommet is first installed into the puncture or otherwise opening that is placed through the tympanic membrane so as to increase the likelihood of a failure mode occurring with respect to transfer of the therapeutic substance delivery device from the ear canal into the middle ear cavity through the opening in the tympanic membrane. In this regard, in an exemplary embodiment, the therapeutic substance delivery device is fed through the hole in the grommet. Again, in keeping with the embodiments where the reservoir is collapsible, the reservoir can be collapsed or otherwise minimized so as to fit through the hole in the grommet.

That said, in an alternate embodiment, the flexible and/or elastic nature of the tympanic membrane can be relied upon to stretch the relatively small opening so that the therapeutic substance delivery device can fit therethrough, and then the flexible and/or elastic nature of the tympanic membrane will cause that hold to reduce in size relative to that which was the case at its maximum diameter during the implantation process.

Still further, in an exemplary embodiment, a lumen or needle or the like can be placed through the opening in the tympanic membrane (the needle or lumen can be the device that is utilized to puncture the tympanic membrane) and that needle or lumen can be utilized as a guide for the therapeutic substance delivery device from one side of the tympanic membrane to the other side of the tympanic membrane, and thus protecting the tympanic membrane from damage during the insertion process. That said, a more larger or beefy structure or significant structure can be utilized as a guide or otherwise to protect the tympanic membrane, such as a tube that is larger than a needle or lumen and that has a thicker wall than a needle or a lumen. In some embodiments, the needle or lumen or tube can be flexible while in other embodiments it is a rigid component.

In an exemplary embodiment, the therapeutic substance transfer device can be angled so that it fits through a given size puncture that is smaller than that which would be the case relative to a scenario where the transfer device was not so angled. In this regard, at least some exemplary embodiments include assembling the therapeutic substance delivery device while such is located in the cavity. By way of example only and not by way of limitation, the therapeutic substance transfer device can be a component that is detachable or otherwise not attached to the reservoir or any other mating component associated therewith, and then fit through the opening in the tympanic membrane or otherwise through the ear canal, and then the reservoir can be placed through the opening and then attached to the therapeutic substance transfer device. Such a process can have utilitarian value with respect to being able to angle or otherwise compress or collapse components of the therapeutic substance delivery device beyond that which would otherwise be the case if the components were connected to one another. Note also that in an exemplary embodiment where the therapeutic substance transfer device is a sponge or a membrane that is flexible or the like, in some embodiments, the transfer device can be compressed. In the same vein, the reservoir can be so compressed. Indeed, in an exemplary embodiment, the components collectively or individually can be located in capsules or the like which restrain or otherwise compress the various components to sizes that would be smaller than that which would be the case if the components were unrestrained or otherwise in their natural relaxed state. In an exemplary embodiment, the capsules can be undone once the components are fit through the opening in the tympanic membrane. In an exemplary embodiment, the capsule could split in half or the capsule could be a fabric or a thin-walled flexible structure with a scene that would rip so that the components therein which spring out of the like. The capsule could be a component that dissolves or otherwise degrades when exposed to a given condition. Still further, mechanical implements can be utilized to compress the components, such as the aforementioned tubes or needles. In an exemplary embodiment, the tubes or needles can be parts of funnel-like devices that compress the components the further the components are moved along the tube or needle. The compressing could be gradual in some embodiments.

It is also noted that in some exemplary embodiments, more than one puncture or opening is made through the tympanic membrane. This can be utilitarian with respect to inserting a device through this second or third puncture that is utilized to position or otherwise guide or otherwise work in the middle ear cavity. By way of example only and not by way of limitation, a needle or lumen can be inserted through the second puncture to apply adhesive or to screw down the distal end (or proximal end, for that matter) of the therapeutic substance. In an exemplary embodiment, an opening can be present for an endoscope or the like. Thus, in an exemplary embodiment, one opening can be utilized to transfer the device into the middle ear (a guidewire can be extended through the opening) and another opening can be utilized for the endoscope.

In an exemplary embodiment, the tympanic membrane is completely removed or partially removed to enable access to the middle ear cavity, and an artificial tympanic membrane is placed in its place. The ossicles can be attached to this new artificial membrane. Note further that in an exemplary embodiment, the tympanic membrane or portion thereof can be removed and the tympanic membrane can then be replaced with the same part that was removed, such as by utilizing healing agents or the like that will enable the membrane that was removed or portions thereof to reattach the other tissue that was not removed.

Method 1700 also includes method action 1730, which includes securing the therapeutic substance delivery device in the middle ear such that the therapeutic substance delivery device delivers therapeutic substance to the cochlea from the middle ear. This can be secured according to any of the exemplary manners detailed herein and/or any other variation thereof or any other arrangement that can have utilitarian value providing that the art enable such.

Accordingly, in view of the above, in an exemplary embodiments of method action 1720, the action is completed by moving all parts of the device through an opening in the tympanic membrane of the person and/or through the ear canal. In an exemplary embodiment, at least 30, 35, 40, 45, 50, 55, 60, 65, 70, 75, 80, 85, 90, 95, or 100%, or any value or range of values therebetween in 1% increments of the components by part and/or by weight and/or by volume are moved through the ear canal and/or through the opening in the tympanic membrane. In some embodiments, the aforementioned values do not include the therapeutic substance while in other embodiments such does include the therapeutic sub stance.

In an exemplary embodiment, the therapeutic substance delivery device is placed into the ear canal and/or the middle ear cavity without any therapeutic substance therein, and then, after it is placed into the ear canal and/or the middle ear cavity, is charged with therapeutic substance. In an exemplary embodiment, the delivery device is placed into the ear canal and/or the middle ear cavity such that the amount of therapeutic substance in the delivery device is more than, less than or equal to 0, 5, 10, 15, 20, 25, 30, 35, 40, 45, 50, 55, 60, 65, 70, 75, 80, 85, 90, 95, or 100% or any value or range of values therebetween in 1% increments of the amount by volume or by weight of the maximum capacity of the therapeutic substance delivery device that will exist at the time that the implantation process is completed.

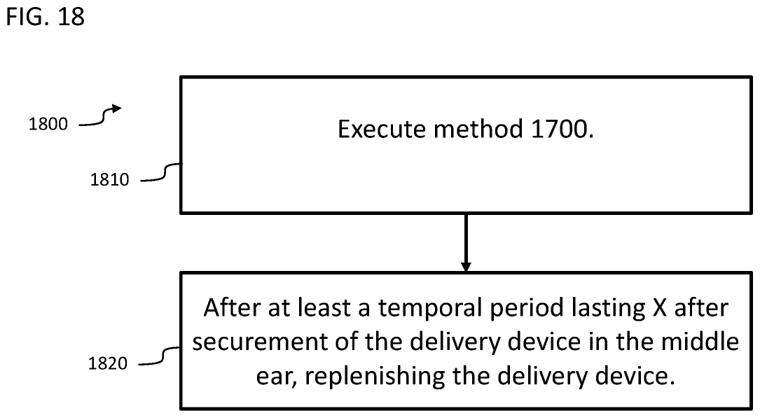

Consistent with the teachings detailed above, embodiments include a reservoir that can be filled and/or refilled after implantation. Again, consistent with the teachings detailed above, embodiments enable a therapeutic substance delivery system that can deliver substance over very long periods of time. This can be achieved by the refilling actions detailed herein or any variation thereof that is enabled by the art. FIG. 18 presents an exemplary flowchart for an exemplary method, method 1800, that includes method action 1810, which includes executing method 1700 or any other method action associated there with in whole or in part. Method 1800 further includes method action 1820, which includes, after at least a temporal period lasting X after securement of the delivery device in the middle ear, replenishing the delivery device with additional therapeutic substance. In an exemplary embodiment, X can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 125, 150, 175, 200, 250, 300, 350, 400, 450, 500 or more, or any value or range of values therebetween in integer increments hours or days or weeks or months.

It is noted that in an exemplary embodiment of the reservoirs or reservoir systems (collectively) that are located in the middle ear, as used herein, can be such that the therapeutic substance can be continuously delivered over a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 125, 150, 175, 200, 250, 300, 350, 400, 450, 500 or more, or any value or range of values therebetween in integer increments hours or days or weeks or months until the therapeutic substance is exhausted or otherwise effectively exhausted, and then in need of replenishment, wherein, upon replenishment, the aforementioned performance features are regained. It is also noted that in an exemplary embodiment, the aforementioned temporal periods can be associated with intermittent but regular application of the therapeutic substance.

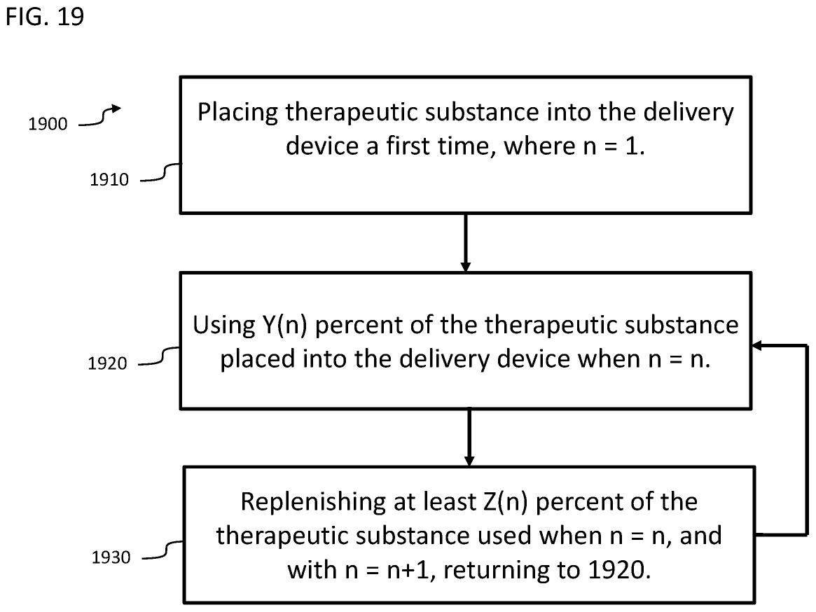

It is noted that method action 1820 can be executed many number of times as the therapeutic substance is utilized. In an exemplary embodiment, FIG. 19 presents an exemplary flowchart for an exemplary method, method 1900, that includes method action 1910, which includes placing therapeutic substance into the delivery device a first time, where n=1. This can be done before the delivery device is implanted into the recipient, during the implantation process and/or after the implantation process (after the area is sealed up). In an exemplary embodiment, this can include placing an amount of therapeutic substance in the delivery device is more than, less than or equal to 0, 5, 10, 15, 20, 25, 30, 35, 40, 45, 50, 55, 60, 65, 70, 75, 80, 85, 90, 95, or 100%, or any value or range of values therebetween in 1% increments of the amount by volume or by weight of the maximum capacity of the therapeutic substance delivery device that will exist at the time that the implantation process is completed.

Method 1900 also includes method action 1920, which includes utilizing Y(n) percent of the therapeutic substance placed into delivery device when n=n (here, 1). Y(n) can be more than, less than, or equal to 5, 10, 15, 20, 25, 30, 35, 40, 45, 50, 55, 60, 65, 70, 75, 80, 85, 90, 95, or 100%, or any value or range of values therebetween in 1% increments. In an exemplary embodiment, there can be a range below which it is time to refill or otherwise add therapeutic substance to the reservoir (akin to not completely depleting a gasoline tank or a heating oil tank, but instead adding fuel to the tanks when the tanks get to a certain level around a certain level of depletion). Indeed, in an exemplary embodiment, the therapeutic substance delivery device can include a sensor that can sense a phenomenon that is indicative of an amount of therapeutic substance remaining in the reservoir or otherwise remaining in the delivery device, whether directly or via a latent variable or the like (e.g., a strain gauge can be located on the reservoir that can be utilized to estimate the amount of fluid left in the reservoir—as the fluid is depleted, the strain on the reservoir will be reduced because the tension on the wall of the reservoir will be reduced, a pressure gauge can be utilized, a flow rate monitor can be utilized to determine the amount of therapeutic substance that has left the reservoir, which can be utilized to estimate the amount that is left if the amount that was originally input was known, etc.).

A temporal schedule can be utilized alternatively and/or in addition to this to determine when to refill or otherwise replenish at least a portion of the therapeutic substance, such temporal schedule can be based on estimated or known performance features of the device (the device is expected to expend a an amount of therapeutic substance per day or per week or per month, etc., and thus the amount of therapeutic substance that has been expended can be estimated, and based thereon a determination can be made when the therapeutic substance will ultimately be depleted or otherwise reduced to a value below which there is no more efficacy or reduced efficacy of a device and/or there is a danger level that the device could run out of therapeutic substance completely, etc.).

In view of the above, it can be seen that method 1900 includes method action 1930, which includes replenishing at least Z(n) percent of the therapeutic substance used when n=n (here, 1), and with n=n+1 (now n=2), returning to method action 1920, which results in the utilization of Y(n) percent of the therapeutic substance (where Y for a given n can be different). Z(n) can be more than, less than or equal to 5, 10, 15, 20, 25, 30, 35, 40, 45, 50, 55, 60, 65, 70, 75, 80, 85, 90, 95 or 100% or any value or range of values therebetween in 1% increments, and as with Y, Z can be different for different n values.

The method of 1900 can be executed for any number of n values, where n can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 125, 150, 175, 200, 250, 300, 350, 400, 450, 500, 600, 700, 800, 900, 1000, 1250, 1500, 2000, 3000, 4000, or 5000 or more, or any value or range of values therebetween in integer increments. In an exemplary embodiment, the time difference between the beginning and the end of method action 1920 and/or the beginning of method action 1920 and the beginning of method action 1930 and/or the beginning of method action 1920 and the end of method action 1930 and/or the end of method action 1920 and the beginning of method action 1930 and/or the end of method action 1920 and the end of method action 1930 can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 80, 90, 100, 125, 150, 175, 200, 250, 300, 350, 400, 450, 500 or more, or any value or range of values therebetween in integer increments hours or days or weeks or months, where these values, as with all values detailed herein, can be different as time progresses and the method actions are repeated.

Accordingly, in an exemplary embodiment, there is a method that results in, after at least one or two or three or four or five or six or seven or eight or nine or ten weeks or months after securement of the delivery device in the middle ear, replenishing the delivery device with additional therapeutic substance.

Further, as can be seen, in an exemplary embodiment there is a method that includes, after any of the aforementioned temporal periods after securement of the delivery device in the middle ear, replenishing the delivery device with additional therapeutic substance at least n separate times (two, three, four, five, six, seven, eight, etc.) separated by temporal periods corresponding to reduction of therapeutic substance due to application of such to the cochlea. In this regard, the temporal periods corresponding to reduction of therapeutic substance can be triggered or otherwise correspond to the utilization of the therapeutic substance for treatment. In an exemplary embodiment, the temporal periods corresponding to reduction of therapeutic substance can correspond to any of the temporal periods detailed herein and/or variations thereof. In an exemplary embodiment, replenishment can be refilling the reservoir to its maximum capacity, or adding an amount that does not result in the reservoir being filled to its maximum capacity.

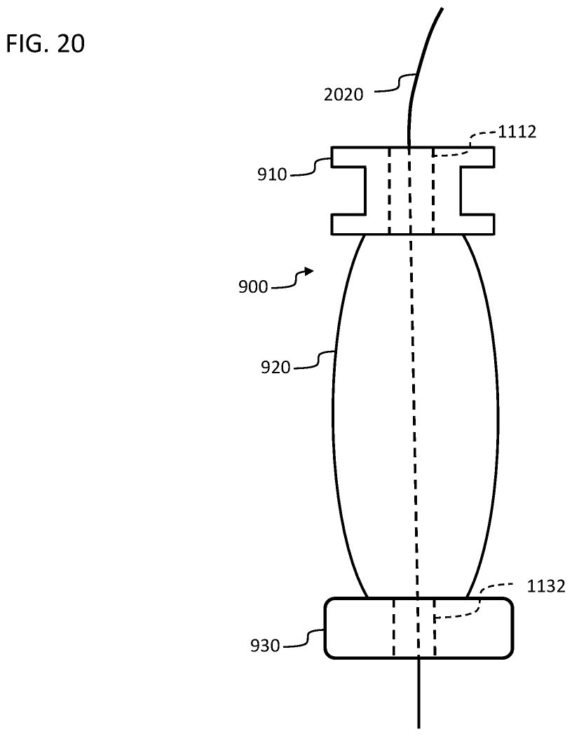

In an exemplary embodiment, method action 1720, the action of inserting the delivery device, is executed by placing a guide device through the opening and then advancing the delivery device along the guide device to a desired location within the middle ear and then removing the guide device while the delivery device remains in the middle ear. In an exemplary embodiment, the guide device can be the guide tube detailed above, where the transfer device is advance through the interior of the tube, which tube can extend through the tympanic membrane and/or through the ear canal into the middle ear cavity. In an exemplary embodiment, the guide device can be a guidewire, such as guidewire 2020 as depicted in FIG. 20. In this exemplary embodiment, the guidewire can extend through the openings 1112 of the grommet and 1132 of the therapeutic substance transfer device 930, or, more accurately, the guidewire 2020 is extended through the tympanic membrane and/or the ear canal such that the tip/distal end of the guidewire becomes placed at the desired location within the middle ear, such as, for example, within the round window niche, and then the therapeutic substance delivery device is advanced along the guidewire until the therapeutic substance delivery device is located at the desired location, and then the guidewire 2020 is removed. In an exemplary embodiment, and end of the guidewire/tip of the guidewire can be placed in and/or located under the round window niche, and the position can be maintained until the device is deployed.

In an exemplary embodiment, the distal end of the guidewire can have a component that at least temporarily establishes a connection between the guidewire and the anatomical structure of the recipient within the middle ear cavity. By way of example only and not by way of limitation, barbs can be located at the end of the guidewire which can grip or otherwise create a friction interface at the anatomical structure, so that the guidewire will be less likely to move from that desired location, all other things being equal. In an exemplary embodiment, the end of the guidewire can be a more flexible and/or collapsible component relative to that which is the case with respect to locations of the guidewire distal therefrom. In an exemplary embodiment, the guidewire can be advanced so that the tip enters the round window niche and then collapses and bends so that the guidewire extends in directions that are somewhat normal or otherwise openly relative to the direction of extension of the guidewire at the locations outside the niche. Because the end of the guidewire bunches in the round window niche, the bunching creates a semi-body like arrangement that will prevent the guidewire from moving relative to that which would otherwise be the case (the wire somewhat “fills” the niche).

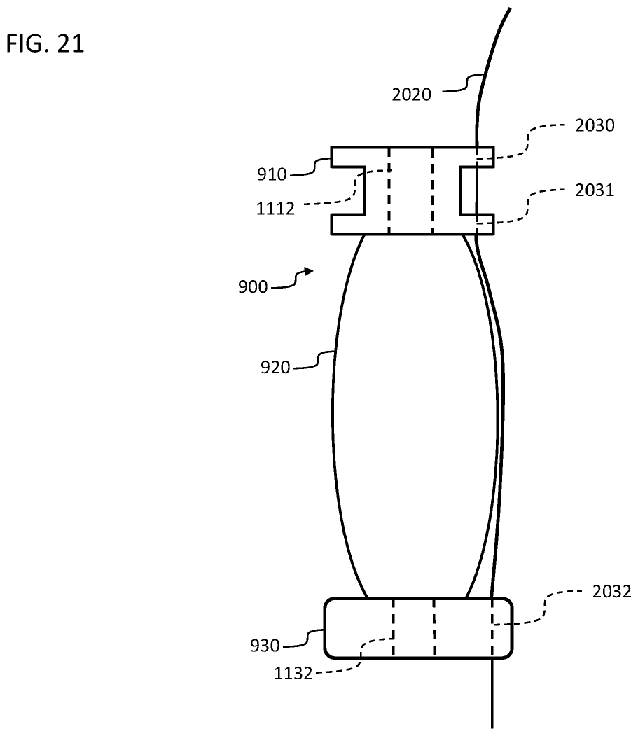

While the embodiments of FIG. 20 depict the guidewire extending through the center of the transfer device, in other embodiments, the guidewire can extend at a location elsewhere, such as off-center, as depicted in FIG. 21. In the embodiment of FIG. 21, there are passages 2030, 2031 and 2032 through which the guidewire 2020 extends. This thus avoids having the guidewire extend through the reservoir and/or through the therapeutic substance movement routes (e.g., the opening 1112 and the opening 1132 and the reservoir, etc.). Note further that outrigger devices can be utilized as well. Indeed, in an exemplary embodiment, there can be guide tubes or guide slots that are located on the sides of the grommet and/or on the sides of the reservoir and/or on the sides of the therapeutic substance transfer device, etc. Note further, that these components can be removable after the delivery device is located in the middle ear. For example, a clip can extend about the grommet and/or about the transfer device 930, which clip has a component that interfaces with the guidewire 2020 after the therapeutic substance transfer delivery device is placed at the desired location, these clips can be removed along with her after the guidewire is retracted. Any arrangement that can enable interface or otherwise functionality with a guiding device, such as a tube or a guidewire etc., can be utilized in at least some exemplary embodiments unless otherwise noted.

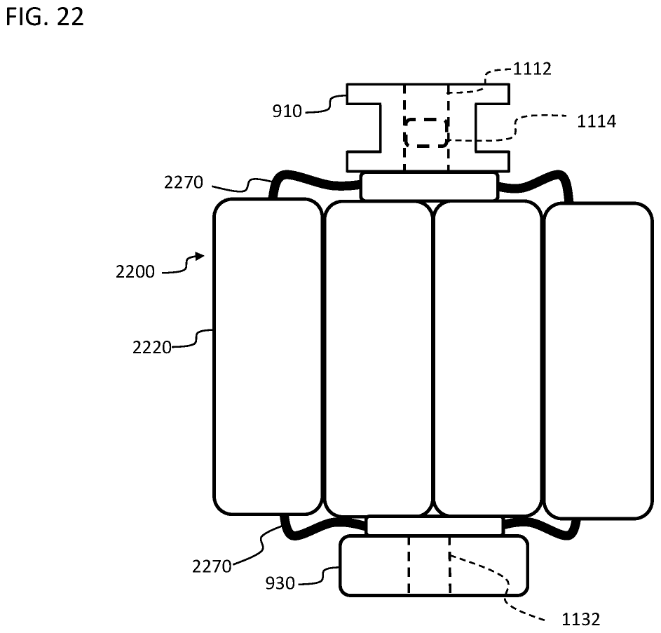

Consistent with the teachings detailed above associated with constructing the therapeutic substance delivery device with in the middle ear cavity piece by piece, FIG. 22 presents an exemplary embodiment that could be placed into the middle ear through a relatively small opening through the tympanic membrane in such a manner. Briefly, the device 2200 of FIG. 22 is somewhat analogous to the Soviet Zond plan to orbit the moon by linking fuel tanks to each other in low Earth orbit and then using the collection to reach the moon. Here, the equivalent of fuel tanks, the cylindrical tanks 2220, can be fed through the puncture in the tympanic membrane one of the time, and then linked together as shown, and then a manifold to 270 (actually two manifolds) can be attached to the separate tanks to place the passageway 1112 into fluid communication with the tanks and to place the tanks into fluid communication with passageway 1132 (or any variations thereof). The tanks could have adhesive on the surfaces thereof so that the tanks would attach to each other and/or to attach to the manifolds, etc. Brackets or clips or spring components or wires or tiny cables can be wrapped around the tanks to hold the tanks or otherwise bunch the tanks. While the embodiment shown in FIG. 22 depicts the tanks arranged in a relatively linear manner, in an alternate embodiment, the tanks can be arrayed in a more haphazard manner.

In this exemplary embodiment, the tanks can be relatively rigid bodies. In some embodiments, the tanks can be flexible, consistent with the balloon embodiments detailed above. In this exemplary embodiment, the tanks can be placed into the middle ear having therapeutic substance therein, and thus could potentially negate any need to initially charge the tanks. Still, in at least some exemplary embodiments, the recharging techniques can be applicable to this embodiment as well.

At least some exemplary embodiments are directed towards apparatus and apparatus that is connected to the tympanic membrane 104, but does not interfere substantially or effectively or at all with respect to functionality of the tympanic membrane and/or the ossicles attached thereto. In an exemplary embodiment, the grommet 910 can articulate relative to the reservoir and/or the component that connects the reservoir thereto. In an exemplary embodiment, a flexible component can be located between the reservoir and the grommet. This flexible component can vibrationally decouple or otherwise reduce any coupling between the grommet and the reservoir that might exist in the absence of this flexible component or the utilization of a component that is less flexible or not flexible at all (the component is rigid/the grommet is rigidly connected to the reservoir 920—as noted above, some or all portions of the reservoir(s) can be rigid—while embodiments described above have been described in terms of a flexible reservoir, in other embodiments, the reservoir can be a rigid reservoir—manifold 2270 can be flexible for example).

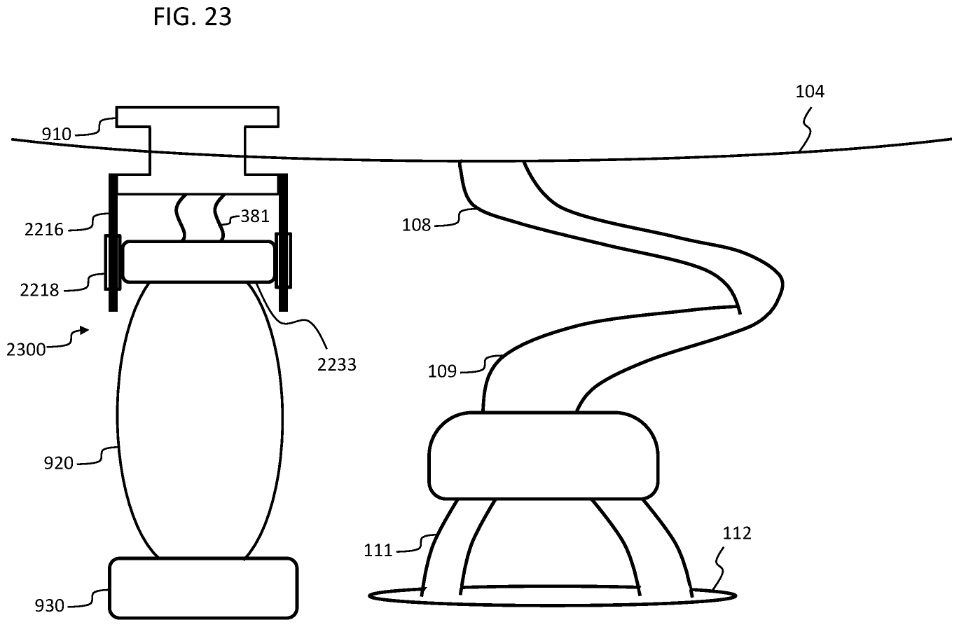

FIG. 23 presents an exemplary embodiment of a therapeutic substance delivery device 2300 that includes a flexible portion that connects the grommet 910 to the rest of the device. Here, flexible component 381 can be a flexible hose or a tube that extends from the grommet to a manifold or a rigid coupling 2233 that places the tube/flexible hose 381 into fluid communication with the reservoir 920. The flexible hose can vibrationally decouple or otherwise reduce the impact of the delivery device on the movement of the tympanic membrane and/or one the operation of the ossicles.

In an exemplary embodiment, a coil or the like that extends from the tympanic membrane with the grommet to the reservoir can be utilized. This coil can vibrationally decouple at least in part the tympanic membrane from the rest of the delivery device. Any device that can minimize damping relative to that which would otherwise be the case can be utilized.

It is noted that while the embodiment depicted in FIG. 23 utilizes a flexible arrangement, in an alternate embodiment, or in addition to this, a telescopic arrangement can be utilized with respect to element 381. In this regard, in an exemplary embodiment, element 381 can be two components that are configured to move relative to one another, such as a male tube and a female tube that receives the male tube. A gasket or the like can be located between the two so as to establish a fluid tight seal. In this exemplary embodiment, as the tympanic membrane moves back and forth and thus moves the grommet, the portion of element 381 that is directly attached to the grommet will move with the grommet and thus with the tympanic membrane, and thus can move relative to the portion of element 381 that is connected to the coupling 2233 and/or to the reservoir 920, thus decoupling in a vibrational manner a portion of the therapeutic substance delivery device from a remainder thereof, and thus freeing the tympanic membrane to move or otherwise enabling the tympanic membrane to move in a less restrictive manner than that which would otherwise be the case.

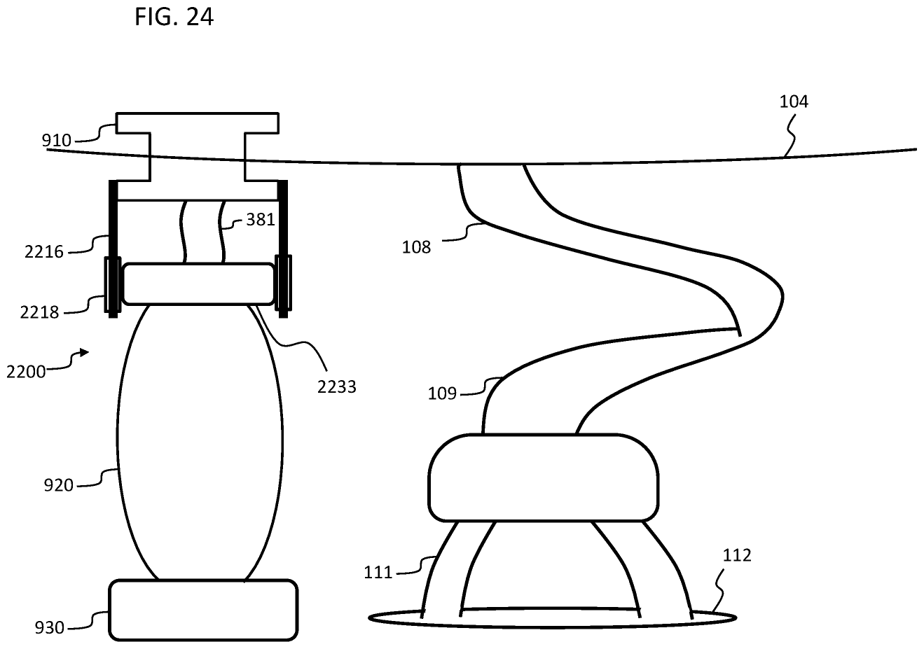

In this exemplary embodiment, a guide structure/support structure is included with the therapeutic substance delivery device. Here, guide rods 2216 are attached to the grommet 910 as can be seen. Two or more guide rods or even one guide rod can be utilized. The guide rods respectively interface with two separate guide tubes 2218 that are located at the sides of the coupling 2233 which couples the flexible (or collapsible/movable) hose 381 to the reservoir 920. Here, the tympanic membrane can move, and move the grommet 910 and the guide rails 2216 with movement thereof, while not being restrained by the remainder of the therapeutic substance delivery device. The movement of the tympanic membrane can be seen by comparing FIG. 23 with FIG. 24. Here, the local portion of the tympanic membrane has moved away from the remainder of the therapeutic substance delivery device, thus pulling the guide rods 2216 away from the coupling 2233. Because the guide rods can move relative to the guide tubes 2218, the movement of the tympanic membrane is not impacted by the rest of the therapeutic substance delivery device. The tympanic membrane can move in the opposite direction (from the location of FIG. 24 back to the location depicted in FIG. 23) again without being restrained or otherwise dampened or slowed by the remainder of the delivery device.

Note also that this embodiment permits the more proximal ends of the therapeutic substance delivery device to be supported by the grommet and/or the tympanic membrane even while permitting the two components to move relative to one another. In this regard, the guide rods 2216 maintain alignment with the various components.

In some other embodiments, an accordion like structure or the like can be utilized as the interface between the grommet and/or the tympanic membrane, etc. and the remainder of the delivery device. Any arrangement that can enable the tympanic membrane to move in a less restrained or unrestrained manner relative to that which would otherwise be the case can be utilized in at least some exemplary embodiments.

In view of the above, it can be seen that in at least some exemplary embodiments of the delivery devices can be utilized with a fully intact ossicles and a fully intact middle ear hearing system. Thus, this device can be utilized without affecting or otherwise effectively affecting the natural hearing or otherwise the natural conduction path from the outer ear to the interior.

Thus, in an exemplary embodiment of the apparatus that enables therapeutic substance delivery according to the teachings detailed herein, where the apparatus includes the reservoir, the device can be configured for contact with a tympanic membrane of the recipient according to any of the devices, systems, and/or methods detailed herein and/or variations thereof or any other manner that can enable such, and the device includes a flexible component between the tympanic membrane and the reservoir that enables the tympanic membrane to move a greater amount than that which would be the case if the component was not flexible. In an exemplary embodiment, with respect to an apples-to-apples comparison, the amount of movement for a given input is at least 5, 10, 15, 20, 25, 30, 35, 40, 45, 50, 60, 65, 70, 75, 80, 90, 100, 125, 150, 175, 200, 250, 300, 350, 400, 450, 500, 600, 700, 800, 900, 1000, 1250, 1500, 1750, 2000, 2500, 3000, 3500, 4000, 5000, 6000, 7000, 8000, 9000, or 10000 percent or more, or any value or range of values therebetween in integer percentile increments relative to that which would be the case if the component was not flexible, all other things being equal. While the just-described embodiment has been described with respect to the flexible connection, it is noted that the aforementioned performance values can also be achieved utilizing the sliding connection or any of the other connections detailed herein that are configured to address movement issues.

Note also that in an exemplary embodiment, the outer surface of the grommet 910 can be lubricated with a like so that the grommet will move with relative ease, or, more accurately, such that the tympanic membrane will move along the longitudinal length of the grommet with ease or more ease relative to that which would otherwise be the case. Thus, in an exemplary embodiment, there can be a substantially rigid structure associated with the therapeutic substance delivery device, where the tympanic membrane slides along the outer surface thereof in a manner that permits the tympanic membrane to move more than that which would otherwise be the case in the absence of the lubricated surface, all other things being equal.

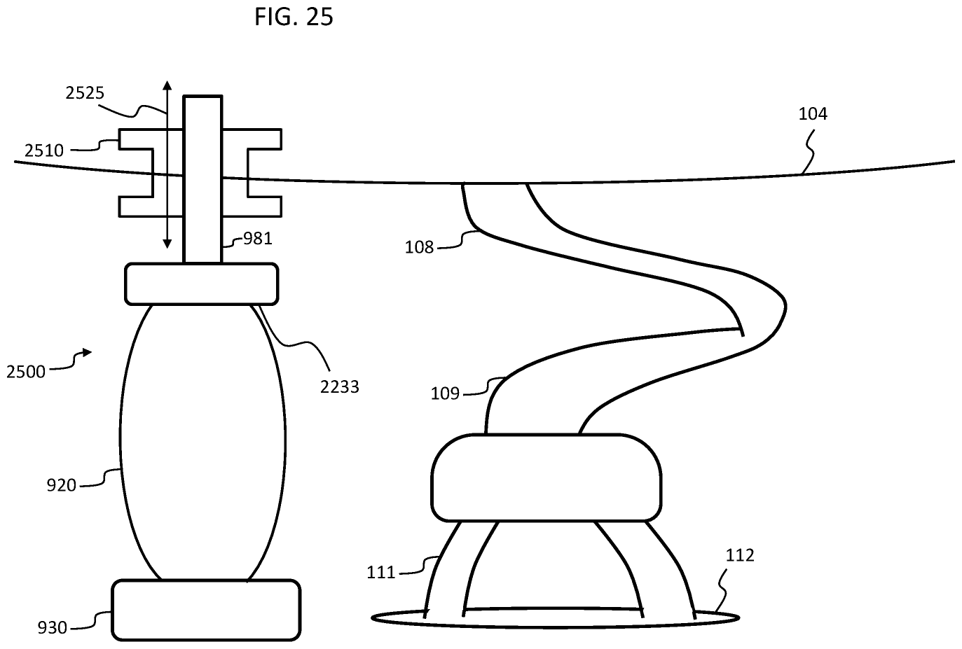

Note also that in at least some exemplary embodiments, the aforementioned feature associated with the male and female components that slide relative to one another can also be applied to the grommet arrangement. In this regard, in an exemplary embodiment, there can be an outer grommet which is configured to not move relative to the tympanic membrane, or more accurately, move as the tympanic membrane moves. Conversely, there can be an inner grommet component which is configured to not move or otherwise remain static while the outer grommet moves along the outer surface thereof. In an exemplary embodiment, the interfacing surfaces can be lubricated to enable such. FIG. 25 depicts such an exemplary arrangement of an exemplary therapeutic substance delivery device 2500, that includes a composite grommet 2510 that includes an inner tube 981 that extends through the outer grommet, as can be seen. In an exemplary embodiment, as the outer grommet moves in an oscillatory manner with respect to arrow 2525, the outer grommet slides along the smooth outer surface of tube 981. Thus, this arrangement provides support for the proximal end of the therapeutic substance delivery device while also permitting the tympanic membrane to move freely relative thereto. Tube 981 permits the reservoir 920 to be replenished via access from the outer ear canal in a manner consistent with the teachings detailed herein. As with some embodiments, a septum or the like can be located within the tube 981. Still further, in some exemplary embodiments, a cap rate valve can be located on tube 981, such as at the topmost portion thereof.

In view of the above, it can be seen that in some exemplary embodiments, there is a therapeutic substance delivery device that is configured so as to limit any damping of the tympanic membrane due to the attachment of the device thereto such that a damping ratio of the tympanic membrane is reduced in some embodiments, or increased in other embodiments, by no more or at least by more than H % relative to that which would be the case in the absence of the attachment, where H can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 56, 70, 75, 80, 81, 82, 83, 84, 85, 86, 87, 88, 89, 90, 91, 92, 93, 94, or 95 percent, or any value or range of values therebetween in 0.1% increments.

Thus, in at least some exemplary embodiments, the therapeutic substance delivery device is a device that does not interfere otherwise does not effectively interfere with normal hearing. In an exemplary embodiment, 1, 2, 3, 4, 5, 6, 7, 8, 9, or 10 days or weeks or months or more or any value or range of values therebetween in one day increments after implantation of the therapeutic substance delivery device, the person retains at least 60, 65, 70, 75, 80, 85, 86, 87, 88, 89, 90, 91, 92, 93, 94, 95, 96, 97, 98, 99 or 100% of his or her hearing at one or more of 500, 750, 1000, 1250, 1500, 1750, 2000, 3000, and/or 4000 Hz when exposed to a pure sine wave at 80 dB relative to that which was the case prior to the implantation, all other things being equal. In an exemplary embodiment, after implantation, the recipient has no hearing impairment that would qualify the recipient to be a disabled person under the Americans with Disabilities Act as that law is interpreted by the pertinent United States government agencies on Sep. 27, 2018.

It is also noted that in at least some exemplary embodiments, the location of the puncture through the tympanic membrane can be utilized to manage the amount of influence that the therapeutic substance delivery device has on the movement of the membrane. By way of example only and not by way of limitation, in an exemplary embodiment where the puncture/grommet is located at the outer periphery of the tympanic membrane, the amount of movement that that location will move relative to portions of the tympanic membrane at the center thereof will be less owing to Pythagoras. Accordingly, some embodiments locate the grommet and/or the puncture at a location that is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 35 or 40 or 45 percent or any value or range of values therebetween in 0.1% increments of the total diameter/maximum diameter of the tympanic membrane from the outer periphery of the tympanic membrane.

In an exemplary embodiment, such as those where the therapeutic substance delivery device extends from contact with the tympanic membrane of the recipient to contact with at least an outer wall of a cochlea of the recipient, with a reservoir in between (and this can be a reservoir to the side), in at least an exemplary embodiment, the reservoir is configured to expand and contract with varying volumes of therapeutic substance therein places effectively no pressure on the membrane (and/or the window in an embodiment where the therapeutic substance delivery device is located proximate thereto and/or in contact there with) due to the expansion and contraction.

As seen above, embodiments are configured to deliver therapeutic substance to the cochlea utilizing a passive delivery system. In an embodiment, the pressurization of the reservoir, which can be due to the elastomeric nature thereof and/or due to a pressure charge therein (e.g., the cylindrical tanks can be charged in a manner analogous to a home water well pump reservoir, with a membrane or piston therein, and a compressible gas on one side and a therapeutic substance in the other, etc.). Any arrangement that can enable passive delivery can be utilized in at least some exemplary embodiments.

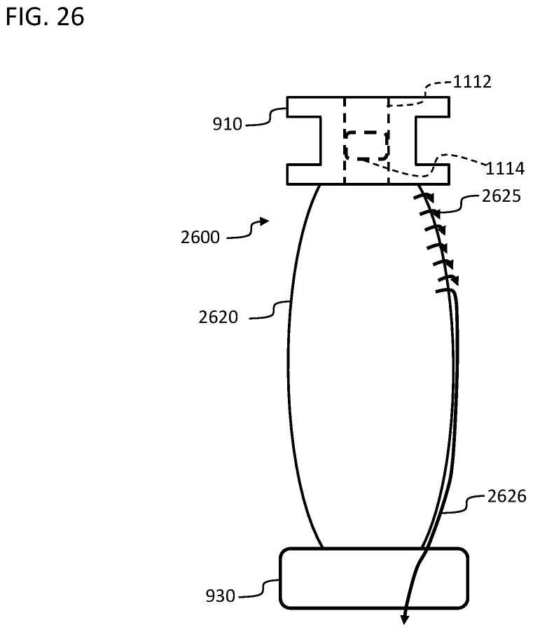

FIG. 26 depicts an alternate exemplary embodiment of a delivery device 2600 where the material of the reservoir 2620 is porous or otherwise where the material is configured to enable the therapeutic substance to seep therethrough to the outside thereof, as represented by arrows 2625. As can be seen, once the therapeutic substance reaches the outer surface of the reservoir 2620 (via, for example, leeching, or by, in other exemplary embodiments, a conduit (or a plurality of conduits) that simply allows the material to move from the inside to the outside, such as akin to a valve faucet (or even without a valve), albeit on a micro-tube scale), the therapeutic substance travels along the outer surface thereof towards the therapeutic substance transfer device 930 as is conceptually represented by arrow 2626. In an exemplary embodiment, this can be achieved via a capillary action and/or by gravity and/or by a pressurized system. The material that establishes the walls of the housing 2620 or otherwise the reservoir 2620 can be an osmosis material or a membrane that can enable such, etc. In an exemplary embodiment, the reservoir 2620, or at least the walls depicted in FIG. 26, can be sheathed inside a second membrane or the like which is not permeable to the therapeutic substance. Thus, in an exemplary embodiment, the therapeutic substance can exude from inside the reservoir to the outer surface thereof, and then, become trapped between that outer surface and the outer sheath, and thus be conducted to the therapeutic substance transfer device 930.

In an exemplary embodiment, therapeutic substance slowly leaches out through the reservoir and then gathers on the surface of the reservoir. Over time, sufficient amounts of therapeutic substance gather on the surface, and then the substances collect and form a fluidic mass that then runs down the outside of the reservoir to the applicator foot. In an exemplary embodiment, the therapeutic substance can gather into droplets or the like and then those droplets run down the side of the reservoir to the applicator foot. In an exemplary embodiment, the applicator foot can soak up the substance and then release the substance to the other side so that the substance reaches the round window or whatever window or whatever trans for medium exists from outside the cochlea to inside the cochlea.

Still further, in an exemplary embodiment, the applicator foot might be dispensed with. In this regard, the reservoir could be configured so that as the therapeutic substance travels down the sides of the reservoir (in channels or in tubes or without such—more on this below) upon reaching the bottom of the reservoir, the therapeutic substance could pool into a drop, and as the therapeutic substance accumulates, the mass/weight of the therapeutic substance overcomes the surface tension and then the drop travels from the reservoir to the round window or oval window or the oval window footplate, etc., and then diffuses into the cochlea therethrough (or the drop drops to whatever transfer medium exists to move the therapeutic substance from outside the cochlea to inside the cochlea).

To be clear, while the embodiments focused on diffusion through the round and/or oval windows, in other embodiments, a catheter or needle or the like extends from therapeutic substance transfer device 930 into the cochlea. A micro-catheter or a plurality of micro-catheters can be utilized.

Note also that in an exemplary embodiment, the therapeutic substance transfer device extends between the round window and the oval window. In this regard, therapeutic substance transfer device can transfer therapeutic substance to the round window and oval window simultaneously and/or in a sequenced manner (such as, for example, the utilization of valving or the like). Therapeutic substance transfer device can be a manifold that shepherds or otherwise guides the therapeutic substance to the two windows. Still further, separate catheters can extend from the reservoir and/or from the transfer device, which catheters separately lead to the separate windows.

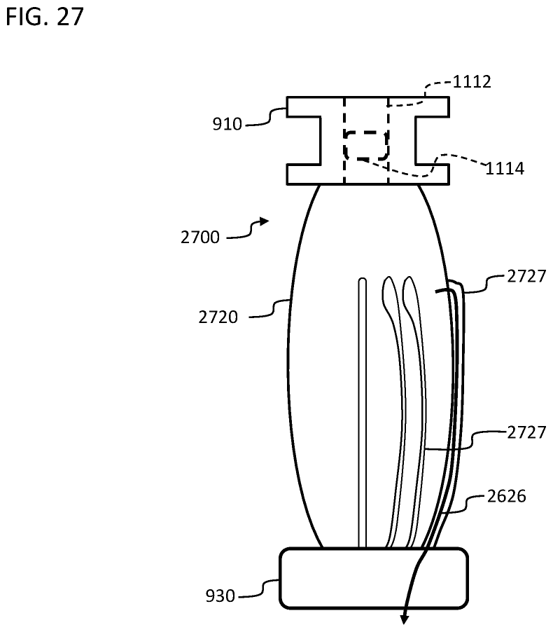

In an exemplary embodiment, guide tubes of the like can be located on the surface of the reservoir 2620. In an exemplary embodiment, various portions of the reservoir wall can be impermeable to the therapeutic substance or otherwise prevents the therapeutic substance from traveling from inside the outside, while other portions thereof that are in fluid communication with these guide tubes enable the transfer of the substance from inside to the outside. FIG. 27 depicts an exemplary therapeutic substance delivery device 2700 that includes a reservoir 2720, that includes delivery tubes 2727. As can be seen, at the tops of the tubes, in at least some of the tubes, there is a bulbous area that permits a provides for a larger face for the therapeutic substance to leach or otherwise travel into the tubes. The size of these bulbous areas can be varied accordingly. Once the therapeutic substance travels outside the reservoir, as represented by arrow 2626, the tubes 2727 guide the therapeutic substance to the transfer device 930. In an alternate embodiment, instead of tubes, open channels are present on the surface of the reservoir, that channel or guide the substance from the outlets towards a distal end of the device/towards the tissue to be treated. In an exemplary embodiment, the puck/footplate can have a cup like device that “collects” the therapeutic substance that is channeled to the puck, and then there are passageways through the puck, or channels about the puck as well, that direct the substance to the tissue. In some embodiments, the channels of the puck/the passageways through the puck are aligned with the channels of the reservoir, while in other embodiments, they are not (the substance simply pools at the bottom, and then reaches the channels/passages). In some embodiments, simple surface tension/adhesion to surface properties is used, and the substance simply runs randomly over the surface of the puck to the tissue. That is, there can be no channels or conduits in some embodiments on the puck (or even on the reservoir in some other embodiments).

The length of the tubes has been depicted as the same, but in other embodiments, the tubes can be varied. Indeed, the tubes of varying lengths can be interleaved with one another.

It is also noted that while the embodiments detailed above have focused on a single therapeutic substance, in some embodiments, the configurations detailed herein can be utilized to provide two or more different therapeutic substances. In an exemplary embodiment, the reservoir can be bifurcated or trifurcated, etc., to have separate volumes inside, in a manner that, in some embodiments, can be analogous to how a gasoline tanker truck having multiple octane grades of gasoline therein is segregated (from the outside, it looks like one tank, but in reality, there are two or three or four or five or six separate tanks therein). In an exemplary embodiment utilizing the tubes, microcontrollers of the like, such as MEMS actuators, can open and/or close the tubes (microvalves, or pinch devices, etc.), to control the amount of a given therapeutic substance relative to another substance, or otherwise vary the temporal locations of delivery of one therapeutic substance relative to another therapeutic substance, etc.

Still further, in an exemplary embodiment, instead of or in addition to active control/valve devices, passive control devices or systems can be utilized. For example, valves, etc., can be opened or closed based on the internal pressure in the reservoir. Further by example, when the reservoir has an internal pressure within a first range, one valve might be open while in other valve might be closed, and then as the pressure is reduced, both the values are opened and/or the first valve can be closed and the second valve can be open, etc. any arrangement that can vary the delivery rates of the therapeutic substance can be utilized in at least some exemplary embodiments.

While embodiments have been directed towards a passive system, in some other embodiments, there can be an active system. In this regard, an electric pump or otherwise an electromagnetic pump can be included within or otherwise attached to the delivery device.

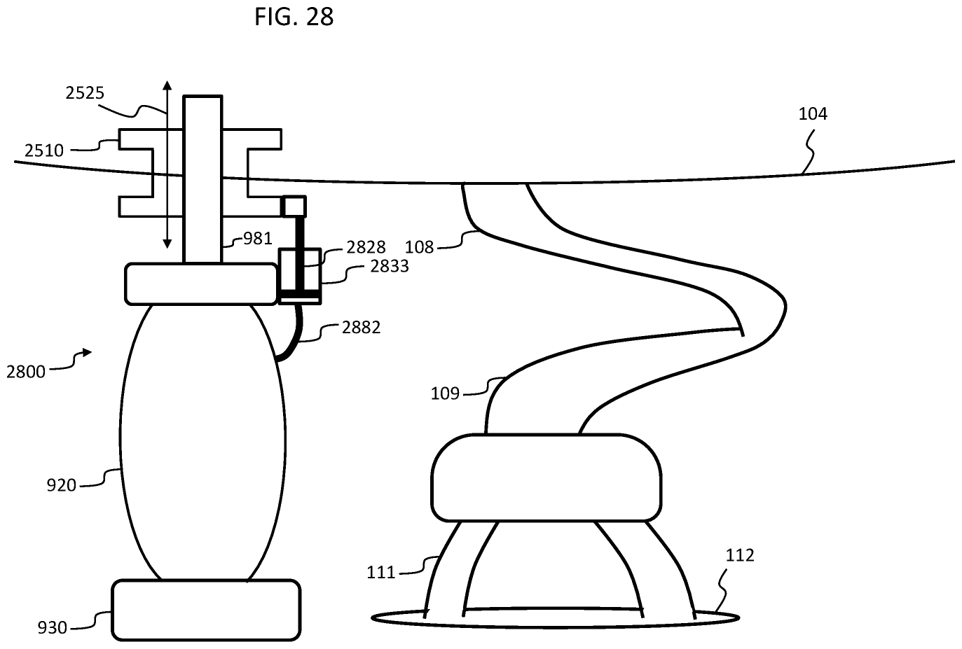

Also, in at least some exemplary embodiments, the movement of the tympanic membrane and/or the ossicles can be utilized as a pump or otherwise to create a pressure imbalance that will move the therapeutic substance from the reservoir to outside the reservoir in a manner beyond that which would otherwise be the case in a passive system. In this regard, FIG. 28 depicts an exemplary therapeutic substance delivery device 2800, that includes a pump arrangement. Here, piston 2828 is mounted to the grommet 2510. As the grommet 2510 moves up and down in the direction of the arrow 2525 with movement of the membrane, the piston 2828 moves within cylinder 2833. The movement of piston 2828 causes pressure to be generated, which is transferred into the reservoir via conduit 2882. This pressurizes the reservoir and thus creates a pressure gradient which can drive the therapeutic substance from the reservoir. Pressure relief valves and/or pressure control valves can be utilized to maintain or otherwise control the pressure in the reservoir.

Thus, in an exemplary embodiment, the micro movement of the tympanic membrane can be utilized to pressurize the system. Still further, in an exemplary embodiment, the micro movement of the tympanic membrane can be utilized to actuate or otherwise start and/or stop the fluid delivery. Accordingly, instead of or in addition to utilizing the movement as a pump or otherwise as a source of energy to transfer of the fluid or otherwise actively move the fluid, in an alternate embodiment, the movement is utilized to simply start and/or stop the transfer. For example, a certain number of movements can result in the beginning of drug delivery and/or a number of movements can result in the end of the drug delivery. For example, it can be like winding a clock. The more that the tympanic membrane moves over time, the more energy is built or otherwise stored in a transducer or the like, and upon the transducer obtaining a level of energy, that initiates or stops an action associated with the therapeutic substance delivery device.

Component 2223 can be a generic support body that simply supports or otherwise provides an interface between the reservoir and the grommet. This can be a machined portion of plastic or titanium, etc. In an alternate embodiment, component 2223 can be a valve. It is also noted that the therapeutic substance transfer device 930 can also be a valve or the like.

Embodiments include control systems which can be microprocessor-based or can be non-microprocessor-based that can control the therapeutic substance delivery device. In an exemplary embodiment, the microprocessor can be included in the delivery device, which microprocessor can be configured or otherwise programmed to control the operation of the delivery device, such as, for example, opening and/or closing valves, controlling the rate of flow, the timing of flow, which therapeutic substances delivered at what time, etc. Alternatively and/or in addition to this, the microprocessor can be in signal communication with sensors or the like, that consents various performance features or other aspects of the delivery device, such as the pressure and/or the amount of therapeutic substance in the device, etc. in an exemplary embodiment, a radio frequency transmitter and/or receiver is also included with the transfer device, which can enable communication from and/or to the transfer device, which can be used to control the operation or otherwise influence the operation of the therapeutic substance transfer device by controlling the valves, etc., and/or which can communicate the status of the transfer device to the outside world.

Note also that in at least some exemplary embodiments, the therapeutic substance transfer device can be placed into signal communication with another implants, such as a middle ear implant under a cochlear implant. In this regard, in an exemplary embodiment, the communication system and/or control system thereof can be utilized to also enable communication with or otherwise enable control of the therapeutic substance delivery device.

Some of the components that can utilize electricity for power can be powered by a small implantable battery. In an exemplary embodiment, the implantable battery can power the delivery device for 1, 2, 3, 4, 5, 6, 7, 8, 9, 10 or more months, or years, or more, or any value or range of values therebetween in 0.1-year increments. Alternatively, and/or in addition to this, power can be provided transcutaneously via an electromagnetic inductance field. That said, in some alternate embodiments, a magnetic field and/or an inductance field can be utilized to control and/or cause the movement of certain components. In an exemplary embodiment, a magnet can be placed outside the skin and/or into the ear canal, which magnetic field thereof can be utilized to activate and/or deactivate components of the therapeutic substance delivery device. Such can be utilized as an alternative and/or in addition to the aforementioned microprocessor or other control regimes. Indeed, in an exemplary embodiment, the therapeutic substance delivery device can be configured so as to operate only when a magnet is located in the ear canal were located behind the ear, etc. Still further, in an exemplary embodiment, the grommet or the like can be utilized to mechanically access the interior or otherwise to access the components of the therapeutic substance delivery device to activate and/or deactivate the device or otherwise influence the operation of the device, etc.

It is noted that in some embodiments, the distal end of the device can be configured to penetrate through the oval and/or round windows into the cochlea to establish fluid communication therewith. Accordingly, in an exemplary embodiment, the delivery device can include, at the end thereof, valves or the like and/or are flanged ports that couple to the cochlea. In some embodiments, the delivery devices extend through the round and oval windows in a manner that seals the round and oval windows between the inner circumference thereof and the delivery devices. Again, additional features of such will be described in greater detail below. That said, it is noted that while some embodiments are directed towards the utilization of intrusive mechanical coupling devices to secure the delivery system to the cochlea, in some alternate embodiments, nonintrusive coupling devices, such as clamps, glues, etc. can be utilized.

An exemplary embodiment includes a holy implanted therapeutic substance delivery device that is implanted entirely in the middle ear. In an exemplary embodiment, there is no transtympanic component. In an exemplary embodiment, the therapeutic substance delivery device can be refilled or otherwise recharged by filling or otherwise providing therapeutic substance directly into the middle ear with drug for a period of time, and allowing the therapeutic substance to saturate into the reservoir, and then transferring that therapeutic substance from the reservoir to the target tissue. In an exemplary embodiment, the apparatus of FIG. 5 can be utilized to transfer therapeutic substance into the middle ear. In an exemplary embodiment, the delivery tube 206 can stop once it enters the middle ear cavity, as opposed to extending all the way to the round window. That said, in an alternate embodiment, the middle ear cavity can be reached utilizing a syringe of the like by going through tissue along the ear canal, but not extending into the ear canal.

It is noted that any reference herein to a therapeutic substance corresponds to a disclosure of an active substance such as an active drug or an active biologic etc., and any disclosure herein to an active substance such as an active drug or the phrase active substance in the generic manner corresponds to a disclosure of an active biologic or a therapeutic substance, etc. Any active pharmaceutical ingredient that can have utilitarian value can be a therapeutic substance. Proteins can be therapeutic substances as well. It is also noted that in an at least some exemplary embodiments, an inactive fluid can be a physiological saline, which can be utilized to convey the therapeutic substance into the cochlea.

In an exemplary embodiment, therapeutic substance includes but is not limited to, any of those detailed above, and can include peptides, biologics, cells, drugs, neurotrophics, etc. Any substance that can have therapeutic features if introduced to the cochlea can be utilized in some embodiments.

Some embodiments include the utilization of the teachings herein and variations thereof to treat otitis media. In an exemplary embodiment, a fast or more powerful elution or otherwise a higher rate of outflow of the therapeutic substance is used to “spray” or “shoot” the substance “sideways” from the reservoir, so that it is sprayed or shot to the walls of the inner ear. That is, some embodiments include ports that open under pressure to permit the substance to spray or be ejected laterally and leave the surface of the reservoir at a direction at an angle (acute, normal) to the tangential surface of the reservoir. In some embodiments, the ports are arrayed about the center of the reservoir, while in other embodiments, the ports can be arrayed in a linear manner along the length of the reservoir, or combinations of the two.

In an exemplary embodiment, the delivery device is configured to be temporarily pressurized while the outlets that permit the substance to leave the reservoir are closed or otherwise limit flow of the substance to a rate lower than that which is normally the case, and then the outlets are opened/the flow rate is permitted to be increased, so that the substance is shot from the reservoir onto the walls of the middle ear. That said, in an alternate embodiment, the puck can be configured to reverse the direction of flow, so that the therapeutic substance flows backwards/into the middle ear cavity away from the cochlea. Thus, some embodiments can be configured for backwards elution. Embodiments can be configured to vary the treatment regime by varying the direction of the flow. That is, there could be a cochlea treatment mode, and an otitis media treatment mode, where the device would alter/vary the flow direction and/or how the substance is applied and/or the rate that the substance is applied (e.g., the rate for otitis media treatment could be at least 2, 3, 4, 5, 6, 7, 8, 9, 10, 11, 12, 13, 14, 15, 20, 25, 30, 35, 40, 45 or 50 or more times the rate for delivery into the cochlea or any values or range of values therebetween.

In another embodiment, the device can be configured to stop flow into the round window niche, for example, and instead let the therapeutic substance accumulate at the foot, and then let the therapeutic substance slosh around in the middle ear so that the substance comes into contact with the tissue in the middle ear (instead of or in addition to the cochlea). In this way, for example, as the recipient moves, the therapeutic substance will slosh around or otherwise coat various tissues in the middle ear.

Again, with respect to the embodiment of treating otitis media, the therapeutic substance could be an antibiotic, for example.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Unless otherwise specified or otherwise not enabled by the art, any one or more teachings detailed herein with respect to one embodiment can be combined with one or more teachings of any other teaching detailed herein with respect to other embodiments, and this includes the duplication or repetition of any given teaching of one component with any like component. Also, embodiments include devices systems and/or methods that explicitly exclude any one or more of a given teaching herein. That is, at least some embodiments include devices systems and/or methods that explicitly do not have one or more of the things that are disclosed herein.

While various embodiments of the present invention have been described above, it should be understood that they have been presented by way of example only, and not limitation. It will be apparent to persons skilled in the relevant art that various changes in form and detail can be made therein without departing from the scope of the invention.

1. An apparatus, comprising:
a refillable therapeutic substance delivery device including a reservoir, the reservoir being configured to be located in a middle ear cavity of a human recipient.

2. The apparatus of claim 1, wherein:
the device is configured such that the reservoir is accessible through a tympanic membrane of the recipient.

3. The apparatus of claim 1, wherein:
the device is configured such that the device is attachable to a tympanic membrane while functioning ossicles are attached thereto.

4. The apparatus of claim 3, wherein:
the device is configured so as to limit any damping of the tympanic membrane due to the attachment of the device thereto such that a damping ratio of the tympanic membrane is reduced by no more than 25% relative to that which would be the case in the absence of the attachment.

5. The apparatus of claim 1, wherein:
the device includes a grommet attachable to a tympanic membrane through which the reservoir can be accessed to refill the reservoir.

6. The apparatus of claim 1, wherein:
the device is configured to deliver therapeutic substance from the reservoir into a cochlea of the recipient across a round window membrane.

7. The apparatus of claim 1, wherein:
the device is configured to extend from a location at least proximate a tympanic membrane to a round window niche of a cochlea.

8. (canceled)

9. An apparatus, comprising:
a refillable therapeutic substance delivery device securable to a round window niche of a recipient.

10. The apparatus of claim 9, wherein:
the therapeutic substance delivery device is refillable while the therapeutic substance delivery device is secured to the round window niche.

11. The apparatus of claim 10, wherein:
the apparatus is configured for implantation into a middle ear of a recipient and to always be in contact with a tympanic membrane of the recipient other than a permanent explantation of the apparatus.

12. The apparatus of claim 9, wherein:
the device includes a reservoir;
the device is configured for contact with a tympanic membrane of the recipient; and
the device includes a flexible component between the tympanic membrane and the reservoir that enables the tympanic membrane to move a greater amount than that which would be the case if the component was not flexible.

13. The apparatus of claim 9, wherein:
the device extends from contact with a tympanic membrane of the recipient to contact at least with an outer wall of a cochlea of the recipient with a reservoir in between; and
the reservoir is configured to expand and contract with varying volumes of therapeutic substance therein without placing any effective pressure on the membrane and the window due to the expansion and contraction.

14. The apparatus of claim 9, wherein:
the device includes a reservoir;
the device includes a silicone body and/or polymer membrane and/or expanded PTFE body in fluid communication with the reservoir; and
the device is configured such that the silicone body and/or polymer membrane and/or expanded PTFE body is in direct contact with a window of a cochlea and/or an anatomical structure attached to the window when the device is implanted in a recipient so that therapeutic substance in the reservoir can travel through the silicone body and/or polymer membrane and/or expanded PTFE body to the window.

15. The apparatus of claim 9, further comprising:
a refill system based on a middle ear pressure equalization tube.

16. An apparatus, comprising:
a means for refillably storing a therapeutic substance; and
a means for delivering a therapeutic substance to a cochlea.

17. The apparatus of claim 16, wherein:
the apparatus is configured to deliver the therapeutic substance to the cochlea via diffuse osmosis.

18. The apparatus of claim 16, wherein:
the apparatus is configured such that the means for delivering the therapeutic substance interfaces with a stapes footplate of the recipient when the apparatus is operationally implanted in the recipient.

19. The apparatus of claim 16, wherein:
the apparatus is configured to extend from a tympanic membrane to the cochlea.

20. (canceled)

21. The apparatus of claim 16, wherein:
the apparatus is configured for contact with a tympanic membrane of the recipient; and
the apparatus includes a means for enabling the tympanic membrane to move a greater amount than that which would be the case in the absence of the means for enabling.

22. The apparatus of claim 16, wherein:
the apparatus is configured to store and deliver a plurality of therapeutic substances in between initial implantation and a first replenishment of the means for storing and/or in between a first replenishment and a second replenishment.

23-30. (canceled)
